(19) United States
(12) Patent Application Publication
(10) Pub. No.: US 2023/0096528 A1
(43) Pub. Date: Mar. 30, 2023

(54) Pharmaceutical Composition for Treatment or Prevention of Multiple Inflammatory disorders

(71) Applicant: AARDVARK THERAPEUTICS, INC., San Diego, CA (US)

(72) Inventors: Tien-Li LEE, San Diego, CA (US); Zhenhuan ZHENG, San Diego, CA (US); Andreas NIETHAMMER, San Diego, CA (US); Anjuli TIMMER, San Diego, CA (US)

(73) Assignee: AARDVARK THERAPEUTICS, INC., San Diego, CA (US)

(21) Appl. No.: 17/845,399

(22) Filed: Jun. 21, 2022

Related U.S. Application Data
(63) Continuation of application No. PCT/US2020/066835, filed on Dec. 23, 2020.
(60) Provisional application No. 62/953,461, filed on Dec. 24, 2019.
(60) Provisional application No. 62/971,202, filed on Feb. 6, 2020.
(60) Provisional application No. 63/022,565, filed on May 10, 2020.
(60) Provisional application No. 63/092,453, filed on Oct. 15, 2020.

(51) Int. Cl. A61K 31/14; A61K 31/19; A61K 9/00; A61P 3/04

(52) U.S. Cl. CPC A61K 31/14; A61K 31/19; A61K 9/0056; A61P 3/04

(57) ABSTRACT

There is disclosed a method for treatment, prevention, and/or slowing of progression for various chronic inflammatory disorder groups including (1) type 2 diabetes group (metabolic syndrome (MET), obesity, hyperglycemia); (2) ARDS (acute respiratory distress syndrome); (3) chronic autoimmune inflammatory disorders (rheumatoid arthritis (RA), lupus, and psoriasis); (4) inflammatory bowel diseases (IBD), such as Crohn's disease and ulcerative colitis; (5) metabolome-mediated diseases (atherosclerosis, hypertension, and congestive heart failure); and (6) hyperphagia disorders such as Prader-Willi Syndrome and other monogenic and syndromic obesity disorders including leptin pathway deficiencies, each comprising administering orally a pharmaceutical composition comprising a denatonium salt. The present disclosure is based on readouts from a series of studies tracking clusters of biomarkers levels to track mediators of inflammatory disorders and mediators of gut-signaling hormones in response to orally administered denatonium salts. There is further disclosed a pharmaceutical composition for treatment and prevention of various inflammatory conditions that can be tracked by pro-inflammatory biomarkers, comprising administering a pharmaceutical composition comprising a denatonium salt. Preferably, the pharmaceutical composition for daily oral administration comprises a denatonium salt delivering a daily total dose of from about 20 mg to about 5000 mg to a human adult BID. Preferably, the denatonium salt is selected from the group consisting of denatonium acetate, denatonium citrate, denatonium maleate and denatonium tartrate.

### RELATED APPLICATIONS

This application is a continuation of International Patent Application No. PCT/US2020/066835, filed Dec. 23, 2020, which claims the benefit of priority to U.S. Provisional Application No. 62/953,461, filed on Dec. 24, 2019, 62/971,202, filed on Feb. 6, 2020, 63/022,565, filed on May 10, 2020, and 63/092,453, filed on Oct. 15, 2020, the entire contents of each of which are incorporated herein by reference.

### TECHNICAL FIELD

The present disclosure provides a method for treatment, prevention, and/or slowing of progression for various chronic inflammatory disorder groups including (1) type 2 diabetes group (metabolic syndrome (MET), obesity, hyperglycemia); (2) ARDS (acute respiratory distress syndrome); (3) chronic autoimmune inflammatory disorders (rheumatoid arthritis (RA), lupus, and psoriasis); (4) inflammatory bowel diseases (IBD), such as Crohn's disease and ulcerative colitis; (5) metabolome-mediated diseases (atherosclerosis, hypertension, and congestive heart failure); and (6) hyperphagia disorders such as Prader-Willi Syndrome and other monogenic and syndromic obesity disorders including leptin pathway deficiencies, each comprising administering orally a pharmaceutical composition comprising a denatonium salt. The present disclosure is based on readouts from a series of studies tracking clusters of biomarkers levels to track mediators of inflammatory disorders and mediators of gut-signaling hormones in response to orally administered denatonium salts. The present disclosure further provides a pharmaceutical composition for treatment and prevention of various inflammatory conditions that can be tracked by pro-inflammatory biomarkers, comprising administering a pharmaceutical composition comprising a denatonium salt. Preferably, the pharmaceutical composition for daily oral administration comprises a denatonium salt delivering a daily total dose of from about 20 mg to about 5000 mg to a human adult BID. Preferably, the denatonium salt is selected from the group consisting of denatonium acetate, denatonium citrate, denatonium maleate and denatonium tartrate.

### BACKGROUND

Over the past 40 years, global levels of obesity have more than doubled. As obesity predisposes to metabolic syndrome and has been linked to coronary heart disease, stroke, type 2 diabetes, certain forms of cancer, and even to greater risk of severe illness and higher risk of death to coronavirus pandemic, this growing epidemic represents one of the most significant current global health challenges. In tandem with the emergence of this problem has been an increase in understanding the pathological mechanisms which link an obese state to the development of disease. Central to these mechanisms is the heightened state of systemic inflammation as a result of obesity, resulting in a multitude of pathologies. Therefore, there is a significant need for treatments and preventives to address appetite and inflammatory signals. The present disclosure addresses this need.

### Inflammatory Diseases

Various inflammatory diseases are currently treated with anti-tumor necrosis factor (TNF) (and anti-interleukin (IL)-6) proteins and antibodies. Such therapeutic proteins are approved for rheumatoid arthritis, polyarticular juvenile idiopathic arthritis (JIA) in children, psoriatic arthritis, lupus, ankylosing spondylitis (AS), chronic plaque psoriasis (Ps), panuveitis, IBD including ulcerative colitis and Crohn's disease, and many other diseases. These biological drugs act by binding and mopping up circulating TNFα (and IL-6) with an antibody or a fusion protein such as etanercept)(Embrel®. However, these anti-TNFα drugs and other biological drugs that indiscriminately bind and mop up inflammatory cytokines have severe side effects. The side effects are caused by inhibition of the vast majority of TNF signaling. As TNF has an immune surveillance function (that is also inhibited by these biological drugs), there is greater susceptibility to infection and decreased immune surveillance, including increased incidence of various infectious diseases and malignancies including leukemias and lymphomas listed on black box warning labels. Therefore, there is a need in the art for more cost-effective small molecule therapeutics that knock down (but not necessarily eliminate) circulating TNF. As protein-based therapeutics cannot be administered orally, there is a need in the art for an oral small molecule agent that is more subtle or self-limiting in their elimination of circulating TNF by preventing TNF production as a pro-inflammatory cytokine instead of mopping up existing and produced TNF indiscriminately.

For example, adalimumab (Humira®) on the U.S. FDA approved label indicates the following side effects of increased risk for serious infections (i.e., including TB and infections caused by viruses, fungi, or bacteria), exacerbation of hepatitis B infection in carriers of the virus, allergic reactions, and various leukemias and lymphomas.

### Metabolic Syndrome

Metabolic syndrome (METS) is a multiplex of factors increasing the risk of the development of type 2 diabetes and cardiovascular disease. METS is a clustering of at least three of the five following medical conditions: (1) visceral obesity; (2) elevated blood pressure; (3) increased blood sugar; (4) high serum triglycerides; and (5) low serum high density lipoprotein (HDL).

According to the International Diabetes Foundation (IDF), metabolic syndrome presents with central obesity and any two of the following: (1) raised triglycerides (TG) of >150 mg/dL (1.7 mmol/L), or specific treatment for increased triglycerides; (2) reduced HDL of <40 mg/dL (1.03 mmol/L) in males<50 mg/dL (1.29 mmol/L in females; (3) raised blood pressure (BP) with systolic>130 or diastolic>85 mm Hg or treatment for hypertension and (4) raised fasting plasma glucose (FPG)>100 mg/dL (5.6 mmol/L) or previous diagnosis of type 2 diabetes.

Metabolic syndrome may also be defined as presentation of hyperinsulinemia and any two of the following: (1) abdominal obesity (waist/hip ration>0.90 or BMI 30 kg/m²), (2) dyslipidemia (TG>1.7 or HDL<0.9 mmol/L) and (3) hypertension (BP>140/90 mm Hg or use of antihypertensive medication). In a clinical study looking at carbohydrate restriction as a first line dietary intervention for METS, the study looked for significance in a group of biomarkers, including the inflammatory biomarkers TNFα, IL-6, and MCP-1 from fasting participants (Al-Sarraj et al., J. Nutrition 139(9):1667-1675, 2009). The study (n=20) found significance for MPC-1, ICAM-1, and TNFα, but not for IL-6.

METS affects 20-25% of the global adult population, including 35% of the U.S. adult population. METS is present in about 60% of U.S. residents aged>50. And METS correlates with a higher frequency of autoimmune diseases. Therefore, there is a need in the art to provide safer and effective METS therapeutics.

### ARDS and Viral Respiratory Infection

Acute respiratory distress syndrome (ARDS) is a life-threatening disease, characterized by acute onset of hypoxia and pulmonary infiltrates, and incited by conditions such as sepsis, pneumonia, trauma, burns, pancreatitis and blood transfusion. ARDS causes diffuse lung inflammation which leads to increased pulmonary vascular permeability, pulmonary edema, and alveolar epithelial injury. The diagnosis of ARDS is made based on the following criteria: (1) acute onset; (2) bilateral lung infiltrates of a non-cardiac origin on chest x-ray or tomographic (CT) scan; and (3) moderate to severe impairment of oxygenation. Severe ARDS carries a mortality rate of 45%. The severity of the ARDS is defined by the degree of hypoxemia, which is calculated as the ratio of arterial oxygen tension to fraction of inspired oxygen (PaO₂/FiO₂). ARDS can be mild, moderate or severe as clarified by the Berlin definition of ARDS, wherein PaO₂/FiO₂ is 200-300 for mild, 100-199 for moderate and <100 for severe.

In general, the development of ARDS can be separated into two phases: an initiator stage followed by an effector stage. The initiator phase of ARDS involves the release of inflammatory mediators (i.e., cytokines; complement and coagulation factors; and arachidonic acid metabolites) which promote systemic inflammation resulting in pulmonary neutrophil sequestration. The second stage, the effector phase, involves the activation of neutrophils with subsequent release of toxic oxygen radicals and proteolytic enzymes, specifically neutrophil elastase (NE). NE has the capacity to injure pulmonary endothelial cells and degrade products of the extracellular matrix, such as elastin, collagen, and fibronectin which comprise the lung basement membrane.

Many diverse forms of ARDS exist with disparate etiologies and courses, although the end-state pathologies of these diverse forms are the same. Examples of clinical events that may precipitate different forms of ARDS include trauma, hemorrhage, diffuse pneumonia, virally induced pneumonia (including, but not limited to COVID-19 and SARS), inhalation of toxic gases, and sepsis. In the case of the 2020 COVID-19 pandemic, it is a viral pneumonia that drives the ARDS observed in many patients requiring critical care. Irrespective of initial cause, ARDS has the following in common: intrapulmonary fluid accumulation and exudates leading to diffuse alveolar damage and impaired gas exchange in the alveoli. What is common (irrespective of the initial cause of the ARDS) downstream is a worsening due to inflammation, fluid release, cell migration and proliferation as well as increases of proinflammatory cytokines.

Viral respiratory infection is generally characterized by an incubation period typically 2-7 days in length, with infected individuals typically exhibiting high fevers, sometimes with accompanying chills, headache, malaise and myalgia. Viral infection of the lungs accounts for approximately 10-15% of ICU admissions in the US per year without a pandemic and is responsible for a significant percentage of deaths from influenza each year without a coronavirus pandemic. The 2020 pandemic from COVID-19 illustrates this course of disease progression. The illness progresses with the onset of a dry, non-productive cough or dyspnea, accompanied by or advancing into hypoxemia. A significant number of cases require intubation and mechanical ventilation. Furthermore, at the peak of respiratory illness, approximately 50% of infected individuals develop leukopenia and thrombocytopenia (MMWR Morb Mortal Wkly Rep. 2003 Mar. 28; 52(12):255-6).

The patterns by which viral load spreads (such as a coronavirus or influenza virus) suggest droplet or contact transmission of a viral pathogen (N. Engl. J. Med. 2003 May 15; 348(20):1995-2005). SARS-1 and -2 have been associated etiologically with a virus, SARS-associated coronavirus (SARS-CoV) is a member of the coronavirus family of enveloped viruses which replicate in the cytoplasm of infected animal host cells. Coronaviruses are generally characterized as single-stranded RNA viruses having genomes of approximately 30,000 nucleotides (Science. 2003 May 30; 300(5624):1394-9). Coronaviruses fall into three known groups; the first two groups cause mammalian coronavirus infections, and the third group causes avian coronavirus infections (J. S. M. Peiris, in Medical Microbiology (Eighteenth Edition), 2012, 587-593). Coronaviruses are believed to be the causative agents of several severe diseases in many animals, for example, infectious bronchitis virus, feline infectious peritonitis virus and transmissible gastroenteritis virus, are significant veterinary pathogens (Viruses. 2019 January; 11(1): 59).

Accordingly, a need exists for an effective treatment for patients diagnosed with SARS, patients infected with an infectious agent associated with SARS, such as patients infected with a SARS-CoV, or patients at imminent risk of contracting SARS, such as individuals that were exposed, or probably will be exposed in the near future, to an infectious agent associated with SARS.

The prior art treatments for ARDS are inadequate. Accordingly, there is an urgent need for an effective treatment of ARDS.

### Metabolome

Intestinal microbiota have gained a lot of attention and disequilibrium of the gut microbiome has been associated with several diseases, depending on which groups of bacteria are increased or decreased. Atherosclerotic disease, with manifestations such as myocardial infarction and stroke, is the major cause of severe disease and death among subjects with the metabolic syndrome. The disease is believed to be caused by accumulation of cholesterol and recruitment of macrophages to the arterial wall and can thus be considered both as a metabolic and inflammatory disease. Since the first half of the 19^{th} century infections have been suggested to cause or promote atherosclerosis by augmenting pro-atherosclerotic changes in vascular cells. However, there is still a need for better ways to early slow down an atherosclerotic change in vascular cells and associated diseases. The present invention provides a method for slowing down atherosclerotic changes in vascular cells by reducing gut signals that support atherosclerotic changes in vascular cells.

### Hyperphagia

The modulation of food behavior, including both control of appetite for some food compositions, and food preferences in favor of less fatty foods or with a lower caloric content, can provide a mechanism for the prevention of the development of metabolic disorders including cardiovascular diseases (Langley-Evans et al., Matern Child Nutr., 1, 142-148, 2005), particularly when food with a high caloric density or rich in fat, particularly saturated fat, is widely available, as happens in our developed societies.

One of the more important signals playing a part in the maintenance of the energy balance and so of body weight is leptin, a circulating protein codified by the ob gene which is mainly expressed in the adipose tissue. Leptin plays a central role in the regulation of energy balance, inhibiting food intake and increasing energy waste (Zhang et al., Nature, 372, 425-432, 1994). This protein circulates in blood in a concentration that is proportional to the size of the fat depots; it passes through the blood-brain barrier by means of a saturable system and exerts most of its effects on energy balance at a central level, through the interaction of the protein with receptors located in hypothalamic neurons and in other regions of the brain (Tartaglia et al., Cell. 83, 1263-1271, 1995).

Animals with defects in the leptin signaling axis, because they do not produce the functional protein or because they express defective forms of its receptor, are characterized by hyperphagia and massive obesity of early appearance, as well as by suffering diabetes, hypothermia and infertility. In humans, congenic defects in the leptin signaling (lack of leptin or of its receptor) are also related to morbid obesity of early appearance (Clement et al., Nature, 392, 398-401, 1998; Montague et al., Nature, 387, 903-908, 1997; Strobel et al., Nat. Genet., 18, 213-215, 1998). In this sense, the use of leptin in the treatment or prevention of diabetes mellitus (WO97/02004) whose direct cause is obesity was proposed. Although it was thought that the short-term anorexigenic role of leptin could contribute to controlling the problem of obesity and related disorders in obese people, unfortunately, leptin administration alone has been ineffective as a practical treatment, in part due to tolerance as well as compensatory upregulation of other pathways mediating hunger and satiety. Long term treatment outcome has remained unsatisfactory.

With age, circulating levels of leptin increase (Matheny et al., Diabetes 1997, 46, 2035-9; Iossa et al., J Nutr. 1999, 129, 1593-6) and there is an impairment in sensitivity to this hormone (Qian et al., Proc. Soc. Exp. Biol. Med. 1998, 219, 160-5; Scarpace et al., Neuropharmacology, 2000, 39, 1872-9). Moreover, high levels of circulating leptin may favor the development of resistance to the anorexigenic effects of this hormone, which leads to perpetuating the development and maintenance of obesity and/or its complications. In fact, there is evidence suggesting that, in rats, leptin resistance would be the main determinant of body weight increase and age-related adiposity [Iossa et al., J. Nutr., 1999, 129, 1593-6]. However, although the concentration of circulating leptin is usually considered to be proportional to body fat mass and this mass usually increases as we grow old, there is evidence that the increase in leptinemia and the development of leptin resistance with age occurs, at least in part, independently of the increase in adiposity (Gabriely et al., Diabetes, 2002, 51, 1016-21).

High leptin circulating levels have been also associated in humans with an increase in the risk of cardiovascular disease [Ren, J. Endocrinol., 2004, 181, 1-10] and development of insulin resistance [Huang et al., Int. J. Obes. Relat. Metab. Disord., 2004, 28, 470-5], and this even independently of body mass index/adiposity.

### SUMMARY

The present disclosure provides a method for treatment, prevention, and/or slowing of progression for various chronic inflammatory disorder groups including (1) type 2 diabetes group (metabolic syndrome (MET), obesity, hyperglycemia); (2) ARDS (acute respiratory distress syndrome); (3) chronic autoimmune inflammatory disorders (rheumatoid arthritis (RA), lupus, and psoriasis); (4) inflammatory bowel diseases (IBD), such as Crohn's disease and ulcerative colitis; (5) metabolome-mediated diseases (atherosclerosis, hypertension, and congestive heart failure); and (6) hyperphagia disorders such as Prader-Willi Syndrome and other monogenic and syndromic obesity disorders including leptin pathway deficiencies, each comprising administering orally a pharmaceutical composition comprising a denatonium salt. The present disclosure is based on readouts from a series of studies tracking clusters of biomarkers levels to track mediators of inflammatory disorders and mediators of gut-signaling hormones in response to orally administered denatonium salts. The present disclosure further provides a pharmaceutical composition for treatment and prevention of various inflammatory conditions that can be tracked by pro-inflammatory biomarkers, comprising administering a pharmaceutical composition comprising a denatonium salt. Preferably, the pharmaceutical composition for daily oral administration comprises a denatonium salt delivering a daily total dose of from about 20 mg to about 5000 mg to a human adult BID. Preferably, the denatonium salt is selected from the group consisting of denatonium acetate, denatonium citrate, denatonium maleate and denatonium tartrate.

The present disclosure provides a method for treatment, prevention and slowing down exacerbation of type 2 diabetes including metabolic syndrome (MET), obesity, and hyperglycemia, comprising administering orally a pharmaceutic composition comprising a denatonium salt, wherein the denatonium salt is selected from the group consisting of denatonium acetate (DA), denatonium citrate, denatonium maleate, denatonium saccharide, and denatonium tartrate. Preferably, the pharmaceutical composition further comprises from about 0.5 g to about 5 g acetic acid. More preferably, the dosage per day of the acetic acid for an adult is from about 1.5 g to about 3 g. Preferably the daily dosage of the denatonium salt for an adult is from about 20 mg to about 5000 mg or from about 5 mg/kg to about 150 mg/kg body weight per day. More preferably, the daily dosage of DA for an adult is from about 50 mg to about 1000 mg. Most preferably, the daily dosage of DA for an adult is from about 60 mg to about 500 mg, or to achieve a concentration in the GI tract of from about 10 parts per billion to about 50 ppm. The daily dose of the denatonium salt is administered once per day, twice per day or three times per day.

The present disclosure provides a method for treatment, prevention and slowing down exacerbation of acute pulmonary inflammatory disorders including ARDS, comprising administering orally a pharmaceutic composition comprising a denatonium salt, wherein the denatonium salt is selected from the group consisting of denatonium acetate (DA), denatonium citrate, denatonium maleate, denatonium saccharide, and denatonium tartrate. Preferably, the pharmaceutical composition further comprises from about 0.5 g to about 5 g acetic acid. More preferably, the dosage per day of the acetic acid for an adult is from about 1.5 g to about 3 g. Preferably the daily dosage of the denatonium salt for an adult is from about 20 mg to about 5000 mg or from about 5 mg/kg to about 150 mg/kg body weight per day. More preferably, the daily dosage of DA for an adult is from about 50 mg to about 1000 mg. Most preferably, the daily dosage of DA for an adult is from about 60 mg to about 500 mg, or to achieve a concentration in the GI tract of from about 10 parts per billion to about 50 ppm. The daily dose of the denatonium salt is administered once per day, twice per day or three times per day.

The present disclosure provides a method for treatment, prevention and slowing down exacerbation of chronic autoimmune inflammatory disorders group of indications selected from the group consisting of rheumatoid arthritis (RA), lupus, and psoriasis, comprising administering orally a pharmaceutic composition comprising a denatonium salt, wherein the denatonium salt is selected from the group consisting of denatonium acetate (DA), denatonium citrate, denatonium maleate, denatonium saccharide, and denatonium tartrate. Preferably, the pharmaceutical composition further comprises from about 0.5 g to about 5 g acetic acid. More preferably, the dosage per day of the acetic acid for an adult is from about 1.5 g to about 3 g. Preferably the daily dosage of the denatonium salt for an adult is from about 20 mg to about 5000 mg or from about 5 mg/kg to about 150 mg/kg body weight per day. More preferably, the daily dosage of DA for an adult is from about 50 mg to about 1000 mg. Most preferably, the daily dosage of DA for an adult is from about 60 mg to about 500 mg, or to achieve a concentration in the GI tract of from about 10 parts per billion to about 50 ppm. The daily dose of the denatonium salt is administered once per day, twice per day or three times per day.

The present disclosure provides a method for treatment, prevention and slowing down exacerbation of chronic IBD group of indications selected from the group consisting of Crohn's Disease, and ulcerative colitis, comprising administering orally a pharmaceutic composition comprising a denatonium salt, wherein the denatonium salt is selected from the group consisting of denatonium acetate (DA), denatonium citrate, denatonium maleate, denatonium saccharide, and denatonium tartrate. Preferably, the pharmaceutical composition further comprises from about 0.5 g to about 5 g acetic acid. More preferably, the dosage per day of the acetic acid for an adult is from about 1.5 g to about 3 g. Preferably the daily dosage of the denatonium salt for an adult is from about 20 mg to about 5000 mg or from about 5 mg/kg to about 150 mg/kg body weight per day. More preferably, the daily dosage of DA for an adult is from about 50 mg to about 1000 mg. Most preferably, the daily dosage of DA for an adult is from about 60 mg to about 500 mg, or to achieve a concentration in the GI tract of from about 10 parts per billion to about 50 ppm. The daily dose of the denatonium salt is administered once per day, twice per day or three times per day.

The present disclosure provides a method for treatment, prevention and slowing down exacerbation of metabolome mediated group of indications selected from the group consisting of atherosclerosis, hypertension, and congestive heart failure (CHF), comprising administering orally a pharmaceutic composition comprising a denatonium salt, wherein the denatonium salt is selected from the group consisting of denatonium acetate (DA), denatonium citrate, denatonium maleate, denatonium saccharide, and denatonium tartrate. Preferably, the pharmaceutical composition further comprises from about 0.5 g to about 5 g acetic acid. More preferably, the dosage per day of the acetic acid for an adult is from about 1.5 g to about 3 g. Preferably the daily dosage of the denatonium salt for an adult is from about 20 mg to about 5000 mg or from about 5 mg/kg to about 150 mg/kg body weight per day. More preferably, the daily dosage of DA for an adult is from about 50 mg to about 1000 mg. Most preferably, the daily dosage of DA for an adult is from about 60 mg to about 500 mg, or to achieve a concentration in the GI tract of from about 10 parts per billion to about 50 ppm. The daily dose of the denatonium salt is administered once per day, twice per day or three times per day.

The present disclosure provides a method for treatment, or slowing down exacerbation of a hyperphagia group of indications selected from the group consisting of Prader-Willi Syndrome and leptin pathway deficiencies, comprising administering orally a pharmaceutic composition comprising a denatonium salt, wherein the denatonium salt is selected from the group consisting of denatonium acetate (DA), denatonium citrate, denatonium maleate, denatonium saccharide, and denatonium tartrate. Preferably, the pharmaceutical composition further comprises from about 0.5 g to about 5 g acetic acid. More preferably, the dosage per day of the acetic acid for an adult is from about 1.5 g to about 3 g. Preferably the daily dosage of the denatonium salt for an adult is from about 20 mg to about 5000 mg or from about 5 mg/kg to about 150 mg/kg body weight per day. More preferably, the daily dosage of DA for an adult is from about 50 mg to about 1000 mg. Most preferably, the daily dosage of DA for an adult is from about 60 mg to about 500 mg, or to achieve a concentration in the GI tract of from about 10 parts per billion to about 50 ppm. The daily dose of the denatonium salt is administered once per day, twice per day or three times per day.

### BRIEF DESCRIPTION OF THE FIGURES

FIG. 1 shows body weight over time with administration of DA compared to vehicle control.

FIG. 2 shows body weight change over time with administration of DA compared to vehicle control.

FIG. 3 shows the body weight change at day 28. There was no statistically significant difference in body weight change at Day 28 between the two experimental groups.

FIG. 4 shows fasting blood glucose levels at day 28. There was no statistically significant difference in blood fasting glucose level at Day 28 between the two experimental groups.

FIG. 5 shows HbA1c levels at day 28. There was no statistically significant difference in blood HbA1c levels at Day 28 between the two experimental groups.

FIG. 6 shows blood HDL levels at day 28. Animals treated with DA at 23.1 mg/kg showed a statistically significant decrease in blood HDL level at Day 28 compared to vehicle-treated animals.

FIG. 7 shows blood LDL cholesterol levels at day 28. There was no statistically significant difference in blood LDL levels at Day 28 between the two experimental groups.

FIG. 8 shows blood total cholesterol level (LDL plus HDL) at day 28. Animals treated with DA at 23.1 mg/kg showed an almost significant decrease in blood total cholesterol levels at Day 28 compared to vehicle-treated animals.

FIG. 9 shows blood insulin levels at day 28. There was no statistically significant difference in blood insulin levels at Day 28 between the two experimental groups.

FIG. 10 shows blood bile acid levels at day 28. There was no statistically significant difference in blood bile acid levels at Day 28 between the two experimental groups.

FIG. 11 shows granulocyte number and percentage at pre-dose and at day 28, Although there was no statistically significant difference, DA-treated animals showed a trend of increasing change in granulocyte number as compared to vehicle-treated controls.

FIG. 12 shows monocyte number and percentage at pre-dose and at day 28. Although there was no statistically significant difference, DA-treated animals showed a trend of increasing change in monocyte number and percentage as compared to vehicle-treated controls.

FIG. 13 shows changes in lymphocyte and white blood cell number at pre-dose and at day 28. Although there was no statistically significant difference, DA-treated animals showed a trend of increasing change in lymphocyte and white blood cell numbers and percentage as compared to vehicle-treated controls.

FIG. 14 shows cumulative food consumption over 28 days. There was no statistically significant difference in food consumption over 28 days between the two experimental groups.

FIG. 15 shows various cytokines analysis in blood at day 28. KC: cytokine-induced neutrophil chemoattractant (CXCL1); MCP-1: monocyte chemoattractant protein-1; MIP-1: macrophage inflammatory protein 1; M-CSF, macrophage colony-stimulating factor; MIP-2: macrophage inflammatory protein 2 (CXCL2); VEGF: vascular endothelial growth factor. KC or CXCL1 and M-CSF showed significant decreases with DA administration.

FIG. 16 shows various cytokines analysis in blood at day 28. IP-10: IFN-γ-Inducible Protein 10 (CXCL10). IL-10 and IL-12 showed significant decreases with DA administration.

FIG. 17 shows various cytokines analysis in blood at day 28. G-CSF: granulocyte colony-stimulating factor; GM-CSF: granulocyte-macrophage colony-stimulating factor; IFNγ: interferon gamma; IL-1α, IL-1β, IL-2 and IL-5. GM-CSF, IFNγ, and IL-5 showed significant decreases with DA administration.

FIG. 18 shows a figure of infiltrating cell counts in air pouch exudates wherein pre-treatment with DA decreased infiltrating cell counts in air pouch exudates following LPS induction in a dose-dependent manner. Animals were pre-treated with DA at 96.4 mg/kg showed significantly lower infiltrating cell count as compared with those pre-treated with vehicle and the lower dose of DA between the results.

FIG. 19 shows a figure of IL-6 levels in air pouch exudates wherein pre-treatment with DA decreased infiltrating cell counts in air pouch exudates following LPS induction in a dose-dependent manner. Animals were pre-treated with DA at 96.4 mg/kg showed significantly lower IL-6 levels as compared with those pre-treated with vehicle and the lower dose of DA between the results.

FIGS. 20-27 show the cytokines levels for G-CSF, Eotaxin, GM-CSF, IFNγ, IL-1a, IL-1b, IL-2, and IL-3, respectively. In this group of cytokines, IL-1b showed significant reduction with the higher dose of DA.

FIGS. 28-35 show the cytokines levels for IL-4, IL-5, IL-7, IL-9, IL-10, IL-12p40, IL-12p70, and IL-13, respectively. In this group of cytokines, IL-10 showed significant reduction with the higher dose of DA.

FIGS. 36-43 show the cytokines levels for IL-15, IL-17, LIF, LIX, IP-10, KC. MCP-1, and MCP-1a, respectively. In this group of cytokines, IL-17 showed significant reduction with the higher dose of DA.

FIGS. 44-50 show the cytokines levels for MIP-1b, MIP-2, M-CSF, MIG, RANTES, VEGF, and TNF-1a, respectively. In this group of cytokines, TNF-1a showed significant reduction with the higher dose of DA.

FIG. 51 shows a summary for the higher dose (orange) and the lower dose (blue) showing significance with an asterisk.

FIG. 52 shows body weight changes during the study period. Treatment with DA showed a significant main effect on body weight (P=0.0052).

FIG. 53 shows body weight at day 10. Animals treated with 69.3 mg/kg DA, BID showed significant effect against DSS-induced body weight loss, as compared to vehicle.

FIG. 54 shows fecal occult blood scores during the study period. Treatment with DA showed a significant main effect on fecal blood status.

FIG. 55 shows fecal consistency score during the study period. Treatment with DA showed significant main effect on fecal consistency.

FIG. 56 shows the combined fecal score during the study period. Treatment with DA showed a significant main effect on combined fecal status.

FIGS. 57 and 58 shows colon weight and length at day 10, respectively. Although no significant difference was observed, treatment with high-dose of DA could counteract DSS-induced decrease in colon weight and length in mice.

FIG. 59 shows spleen weight at day 10. Although no significant effect was observed, treatment with high-dose of DA showed a trend to counteract DSS-induced spleen weight loss in mice.

FIG. 60 shows changes a phylum levels wherein week 4 showed>95% confidence changes in the microbiome at the phylum level for the following: Treatment increased proteobacteria*, verrucomicrobia*, cyanobacteria*. Treatment decreased Bacteroidetes, firmicutes*, deferribacteres and spirochetes*. (*significant differences from control or time 0).

FIG. 61 shows significant differences for treatment versus control at a family level.

FIG. 62 shows a principal coordinate analysis plot.

FIG. 63A-B show a significant enrichment in the pathways for biosynthesis of unsaturated fatty acids upon 4-week DA treatment (upper panel: individual data; lower panel: group data).

FIG. 64A-B show a significant enrichment in the pathways for metabolism of arachidonic acid upon 4-week DA treatment (upper panel: individual data; lower panel: group data).

FIG. 65A-B show a significant enrichment in the pathways for metabolism of cofactors and vitamins upon 4-week DA treatment (upper panel: individual data; lower panel: group data).

FIG. 66A-B show a significant enrichment in pathways for lysine degradation upon 4-week DA treatment (upper panel: individual data; lower panel: group data).

FIG. 67 shows a significant enrichment in pathways for glycolysis and gluconeogenesis upon 4-week DA treatment (group data).

FIG. 68 shows a significant enrichment in phosphatidylinositol signaling upon 4-week DA treatment (group data).

FIG. 69A-B show a significantly decreased signaling for arginine and ornithine metabolism upon 4-week DA treatment (upper panel: individual data; lower panel: group data).

FIGS. 70A-C show graphs comparing biomarkers across many studies by family, showing decreased mean percentages.

In FIG. 71, it should be noted that clusters of multiple biomarkers predict effectiveness for each disease indication and that grouping is shown in FIG. 71.

FIGS. 72 and 73 shows cytokine profiles in lung lavage fluids from the data in Examples 7 and 8, respectively.

FIG. 74 shows DA treatment significantly reduced body weight gain at day 57 in DIO mice as compared to vehicle and CQL.

FIG. 75A shows that at Day 14, treatment with DA significantly reduced daily food intake in DIO mice as compared to vehicle.

FIG. 75B shows that treatment with DA significantly increased daily water intake at Day 28, while treatment with CQL significantly decreased daily water intake, as compared to vehicle, both from Example 9.

FIG. 76 shows that treatments with DA and CQL significantly reduced serum HbA1c level at Day 28, but considerably increased the HbA1c level at Day 56 in DIO mice.

FIG. 77 shows that treatments with DA significantly reduced serum insulin level at Day 28 as compared to vehicle control in DIO mice.

In FIG. 78 although no significant difference was observed, treatment with DA resulted in noticeable decrease in serum LDL levels at days 28 and 56 as compared to vehicle controls.

FIG. 79 shows that treatments with DA significantly increased serum GLP-1 levels in DIO mice at Days 7 and 56 as compared to vehicle control.

FIG. 80 shows that treatments with DA significantly increased serum GLP-2 levels in DIO mice at Day 56 as compared to vehicle control.

FIG. 81 shows that treatments with DA significantly increased serum CCK levels in DIO mice at Day 56 as compared to vehicle control.

FIG. 82 shows that treatments with DA significantly increased serum PYY levels in DIO mice at Day 56 as compared to vehicle control.

FIG. 83 shows treatment with DA significantly decreased serum glucose levels in ob/ob mice.

FIG. 84 shows that treatments with DA significantly lowered serum triglyceride levels as compared to vehicle control in ob/ob mice.

FIG. 85 shows that treatments with DA significantly increased serum bile acids levels as compared to vehicle control in ob/ob mice.

FIG. 86 shows that treatments with DA significantly lowered serum LDL levels as compared to vehicle control in ob/ob mice.

### DETAILED DESCRIPTION

The present disclosure provides a method for treatment, prevention, and/or slowing of progression for various chronic inflammatory disorder groups including (1) type 2 diabetes group (metabolic syndrome (MET), obesity, hyperglycemia); (2) ARDS (acute respiratory distress syndrome); (3) chronic autoimmune inflammatory disorders (rheumatoid arthritis (RA), lupus, and psoriasis); (4) inflammatory bowel diseases (IBD), such as Crohn's disease and ulcerative colitis; (5) metabolome-mediated diseases (atherosclerosis, hypertension, and congestive heart failure); and (6) hyperphagia disorders such as Prader-Willi Syndrome and other monogenic and syndromic obesity disorders including leptin pathway deficiencies, each comprising administering orally a pharmaceutical composition comprising a denatonium salt. The present disclosure is based on readouts from a series of studies tracking clusters of biomarkers levels to track mediators of inflammatory disorders and mediators of gut-signaling hormones in response to orally administered denatonium salts. The present disclosure further provides a pharmaceutical composition for treatment and prevention of various inflammatory conditions that can be tracked by pro-inflammatory biomarkers, comprising administering a pharmaceutical composition comprising a denatonium salt. Preferably, the pharmaceutical composition for daily oral administration comprises a denatonium salt delivering a daily total dose of from about 20 mg to about 5000 mg to a human adult BID. Preferably, the denatonium salt is selected from the group consisting of denatonium acetate, denatonium citrate, denatonium maleate and denatonium tartrate.

The present disclosure is based on a discovery of (1) a cluster of surprising results from what started as a weight loss in vivo study in a predictive ob/ob obesity mouse model with a denatonium salt and placebo controls. The data from several studies in various in vivo models showed that orally administered denatonium salt with an organic acid anion show treatment efficacy and showed significant anti-inflammatory effects first by measuring inflammatory cytokines in the blood and other fluids (e.g., air pouch exudates and lung lavage fluids) as biomarkers and then gut signaling peptides. The methods of treatment that oral administration (but not intravenous administration) provided data showing efficacy for methods of treatment, prevention and slowing down disease progression in indications including metabolic syndrome (METS), obesity (inflammatory mediated), ARDS, rheumatoid arthritis (RA), lupus, and psoriasis (Examples 1 and 2); (2) an in vivo study in a dextran sulfate sodium (DSS)-induced colitis in a mouse model showing treatment and prevention efficacy in indications including inflammatory bowel diseases (IBD), mainly comprising ulcerative colitis and Crohn's disease (Example 3); and (3) a four week microbiome study in mice fed a high fat diet showing treatment and prevention efficacy for atherosclerosis, hypertension, and congestive heart failure (Example 4 and below). A cluster of proinflammation-indicating cytokines measured achieved significant differences between drug administered mice and control mice. Weight loss showed strong trends to in vivo efficacy with DA administration but was not similarly statistically significant.

The cytokine data provided herein show in the inflammatory bowel disease model (Example 3), and in an air pouch model for inflammatory diseases, that the study drug, DA, did exhibit therapeutic activity in three areas: (1) to treat or prevent METS, (2) to treat or prevent general inflammatory diseases including autoimmune diseases; (3) to treat inflammatory bowel diseases including Crohn's Disease and ulcerative colitis; and (4) to treat cardiovascular diseases such as atherosclerosis, hypertension and congestive heart failure from microbiome data. Therefore, the data achieved in these studies does have a story to tell and the story is that a denatonium salt pharmaceutical composition shows safety and efficacy to (1) treat or prevent METS; (2) treat obesity and effect weight loss; (3) treat autoimmune inflammatory conditions rheumatoid arthritis (RA) lupus, and psoriasis; (4), treat Crohn's Disease and inflammatory bowel disease (IBD); and (5) treat or slow disease progression for cardiovascular diseases of atherosclerosis, hypertension and congestive heart failure. Preferably, the denatonium salt is selected from the group consisting of denatonium acetate, denatonium citrate, denatonium maleate and denatonium tartrate. More preferably, the denatonium salt for treating the foregoing listed indication is administered orally from about 25 mg to about 500 mg per day to an adult BID.

In addition, the Example 2 study provided surprising results of statistical significance in reducing IL-5 production, which indicates the effectiveness of the present pharmaceutical composition of denatonium salts including DA in treating ARDS.

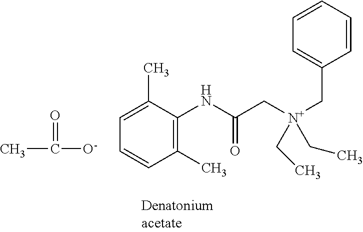

This example describes the synthesis of denatonium acetate (DA).

### Step 1: Synthesis of Denatonium Hydroxide from Lidocaine

To a reflux apparatus add 25 g of lidocaine, 60 ml of water and 17.5 g of benzyl chloride with stirring and heating in 70-90° C. The solution needs to be heated and stirred in the before given value for 24 h, the solution needs to be cooled down to 30° C. The unreacted reagents are removed with 3×10 mL of toluene. With stirring dissolve 65 g of sodium hydroxide into 65 mL of cold water and add it to the aqueous solution with stirring over the course of 3 h. Filter the mixture, wash with some water and dry in open air. Recrystallize in hot chloroform or hot ethanol.

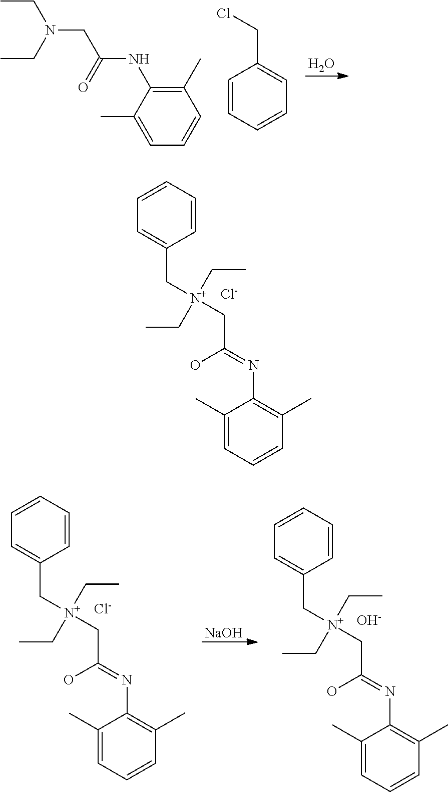

### Step 2: Preparation of Denatonium Acetate from Denatonium Hydroxide

To a reflux apparatus 10 g of denatonium hydroxide (MW: 342.475 g/mol, 0.029 mol), 20 mL of acetone, and 2 g of acetic acid glacial (0.033 mol) dissolved in 15 mL of acetone is added, the mixture is stirred and heated to 35° C. for 3 h. Then evaporated to dryness and recrystallized in hot acetone.

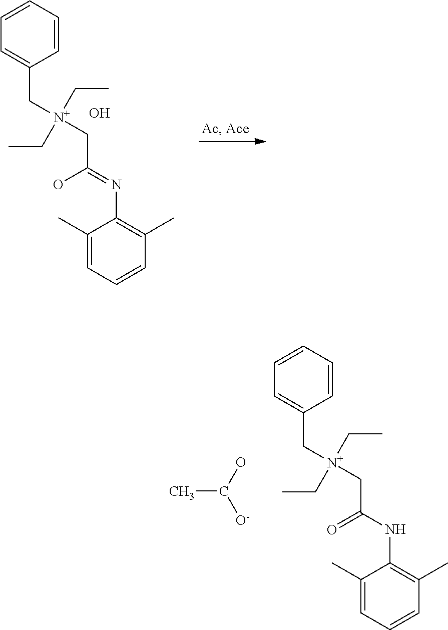

### Formulation of DA Tablet

This provides an immediate release 50 mg granule formulation of denatonium acetate monohydrate (DA) as a free base as an immediate gastric release oral pharmaceutical formulation.

Table 1 shows qualitative and quantitative formulation composition of DA.

| | | | | | Limits based | |
|---|---|---|---|---|---|---|
| | | | | | on IID | |
| | | | | | Max | |
| | | | | DA | Potency | |
| | Quality | | Quantity | capsule- | for Unit | |
| | Stan- | Func- | (%) | 50 mg | Dose | Ref- |
| Ingredient | dard | tion | w/w | (mg/cap) | (mg) | erence |
| Denatonium | In- | API | 23.55 | 59.03 | N/A | N/A |
| acetate | house | | | (20 mg | | |
| mono- | | | | Den- | | |
| hydrate | | | | atonium | | |
| | | | | base) | | |
| Povidone | USP | Binder | 2.36 | 5.90 | 61.5 | Oral— |
| (KOLLIDON | | | | | | Capsule |
| 30) | | | | | | |
| Sugar | NF | Sub- | 68.85 | 172.57 | 314.13 | Oral— |
| Spheres | | strate | | | | Capsule |
| (VIVAPHA | | | | | | |
| RM ® | | | | | | |
| Sugar | | | | | | |
| Spheres | | | | | | |
| 35-45) | | | | | | |
| Hypro- | USP | Binder | 3.64 | 9.14 | 150 | Oral— |
| mellose | | | | | | Capsule |
| (Methocel | | | | | | |
| E5 | | | | | | |
| Premium | | | | | | |
| LV | | | | | | |
| Hydroxy- | | | | | | |
| propyl | | | | | | |
| Methyl- | | | | | | |
| cellulose) | | | | | | |
| Talc | USP | Anti- | 1.09 | 2.74 | 14 | Oral— |
| (MicroTalc | | tacking | | | | Capsule, |
| MP 1538 | | agent | | | | coated |
| USP Talc) | | | | | | pellets |
| Talc (extra | USP | Flow | 0.50 | 1.25 | 284.38 | Oral— |
| granular) | | aid | | | | Capsule |
| (MicroTalc | | | | | | |
| MP 1538 | | | | | | |
| USP Talc) | | | | | | |
| Total weight of beads | | | 250.62 | | N/A | N/A |
| Hard | USP | Capsule | N/A | 73.3 | 107 | Oral— |
| Gelatin | | shell | | | | Capsule |
| Capsule | | | | | | |
| Shells; | | | | | | |
| Cap: | | | | | | |
| White | | | | | | |
| Opaque: | | | | | | |
| Body: | | | | | | |
| White | | | | | | |
| Opaque; | | | | | | |
| Size: 1 | | | | | | |

| | | | |
|---|---|---|---|
| Total weight of Filled Capsule | 323.9 | N/A | N/A |

IID, the Inactive Ingredient Database;
API, active pharmaceutical ingredient;
USP, the US Pharmacopeia;
NF, the National Formulary
* Solvents such as Ethyl Alcohol USP 190 Proof (190 Proof Pure Ethyl Alcohol) and purified water (USP) were used for the preparation of drug solution and seal coating dispersion, but are removed during the manufacturing process.

The detailed manufacturing steps are described below.

### 1. Drug Layering Process—Drug Layered Pellets

Drug layering process was performed in a Fluid bed granulator equipped with the rotor insert (rotor granulator). Drug solution was prepared by solubilizing Povidone K30 (Kollidon 30) and Denatonium Acetate in ethyl alcohol. The drug solution was sprayed tangentially on to the bed of sugar spheres (35/45 mesh) moving in a circular motion in the rotor granulator. The final drug loaded pellets were then dried for ten (10) minutes in the rotor granulator, discharged and screened through a #20 mesh.

### 2. Seal Coating Process—Seal Coated Pellets

Seal coating dispersion was prepared by separately dissolving Hypromellose E5 in a mixture (1:1) of ethyl alcohol and purified water until a clear solution was obtained. The remaining quantity of ethyl alcohol was then added to the above solution followed by talc. The dispersion was mixed for 20 minutes to allow for uniform dispersion of talc. The seal coating dispersion was sprayed tangentially on to the drug loaded pellets to achieve 5% weight gain. The seal coated pellets were then dried for five (5) minutes in the rotor granulator, discharged and dried further in a tray dryer/oven at 55° C. for 2 hours. The seal coated pellets were then screened through a #20 mesh.

### 3. Final Blending—Denatonium Immediate Release (IR) Pellets

The seal coated pellets were blended with talc screened through mesh #60 using a V-Blender for ten (10) minutes and discharged. The blended seal coated beads, Denatonium IR Pellets, were used for encapsulation.

### 4. Encapsulation—Denatonium Capsules, 50 mg

The Denatonium IR pellets, 50 mg, were filled into size 1, white opaque hard gelatin capsules using an auto capsule filling machine. Capsules were then passed through an in-line capsule polisher and metal detector. In-process controls for capsule weight and appearance was performed during the encapsulation process. Acceptable quality limit (AQL) sampling and testing was performed by Quality Assurance (QA) on a composite sample during the encapsulation process. Finished product composite sample was collected and analyzed as per specification for release testing.

5. Packaging—Capsules, 50 mg-30 Counts

The 50 mg capsules were packaged in 30 counts into 50/60 cc White HDPE round S-line bottles with 33 mm White CRC Caps. The bottles were torqued and sealed using an induction sealer.

### Nexus of Biomarkers to Disease Indications

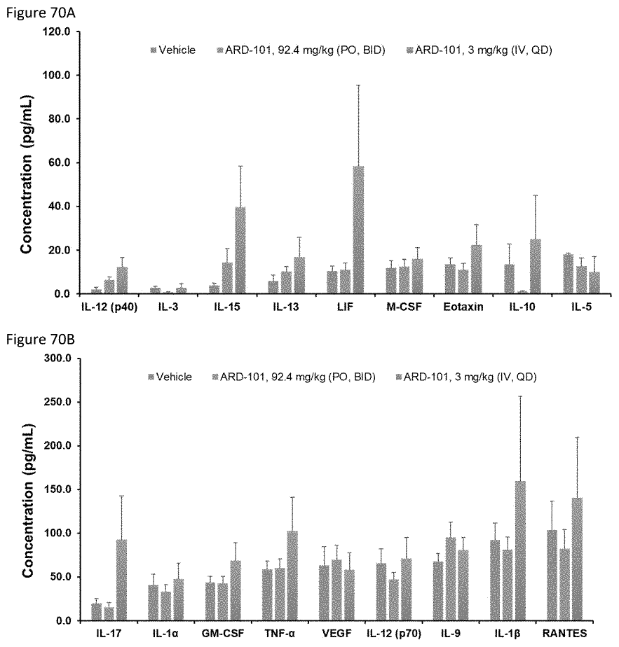

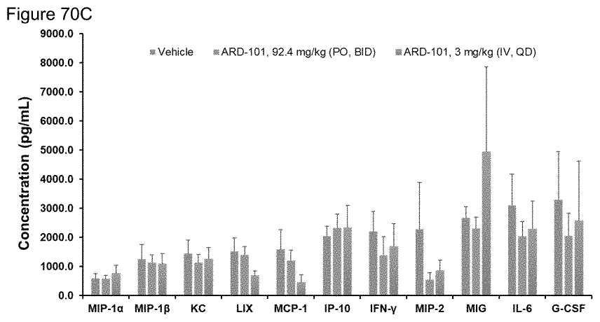

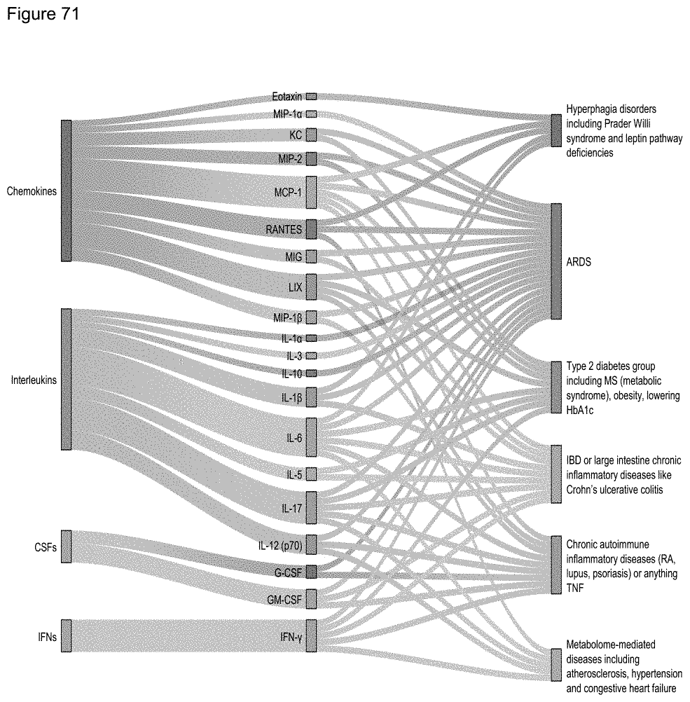

The many examples provided herein show the effect of the denatonium salts on various in vivo and in vitro models of various disease indications. In addition, blood samples were taken from the tested (and control) animals and various biomarkers were measured and compared. FIGS. 70A-C show graphs comparing biomarkers across many studies. Table 2 groups the biomarkers by family, shows decreased mean percentages and shows which disease indications are impacted and predicted by each biomarker. It should be noted that clusters of multiple biomarkers predict effectiveness for each disease indication and that grouping is shown in FIG. 71.

TABLE 2

| | | Decreased Percentage | |
|---|---|---|---|
| | | with PO 92.4 mg/kg DA | |
| Family | Member | BID Compared to Vehicle | Nexus to Indications |
| Chemokines | Eotaxin | −18.6% | Hyperphagia disorders such as Prader- |
| | | | Willi Syndrome and other monogenic and |
| | | | syndromic obesity disorders including |
| | | | leptin pathway deficiencies |
| | MIP-2 | −76.3% | Type 2 diabetes group (metabolic |
| | | | syndrome, obesity, hyperglycemia) |
| | | | ARDS |
| | KC | −21.6% | Type 2 diabetes group (metabolic |
| | | | syndrome, obesity, hyperglycemia) |
| | | | ARDS |
| | MCP-1 | −24.2% | Type 2 diabetes group (metabolic |
| | | | syndrome, obesity, hyperglycemia) |
| | | | Chronic autoimmune inflammatory |
| | | | disorders (rheumatoid arthritis, lupus, and |
| | | | psoriasis) |
| | | | Inflammatory bowel diseases, such as |
| | | | Crohn’s disease and ulcerative colitis |
| | | | ARDS |
| | | | Hyperphagia disorders such as Prader- |
| | | | Willi Syndrome and other monogenic and |
| | | | syndromic obesity disorders including |
| | | | leptin pathway deficiencies |
| | MIP-1α | −3.4% | ARDS |
| | MIP-1β | −10.2% | ARDS |
| | | | Chronic autoimmune inflammatory |
| | | | disorders (rheumatoid arthritis, lupus, and |
| | | | psoriasis) |
| | RANTES | −20.6% | ARDS |
| | | | Hyperphagia disorders such as Prader- |
| | | | Willi Syndrome and other monogenic and |
| | | | syndromic obesity disorders including |
| | | | leptin pathway deficiencies |
| | | | Metablome-mediated diseases |
| | | | (atherosclerosis, hypertension, and |
| | | | congestive heart failure) |
| | LIX | −7.3% | Type 2 diabetes group (metabolic |
| | | | syndrome, obesity, hyperglycemia) |
| | | | ARDS |
| | | | Inflammatory bowel diseases, such as |
| | | | Crohn’s disease and ulcerative colitis |
| | | | Chronic autoimmune inflammatory |
| | | | disorders (rheumatoid arthritis, lupus, and |
| | | | psoriasis) |
| | MIG | −13.2% | ARDS |
| | | | Inflammatory bowel diseases, such as |
| | | | Crohn’s disease and ulcerative colitis |
| CSFs | GM-CSF | −2.3% | Chronic autoimmune inflammatory |
| | | | disorders (rheumatoid arthritis, lupus, and |
| | | | psoriasis) |
| | | | Inflammatory bowel diseases, such as |
| | | | Crohn’s disease and ulcerative colitis |
| | | | ARDS |
| | G-CSF | −37.5% | Chronic autoimmune inflammatory |
| | | | disorders (rheumatoid arthritis, lupus, and |
| | | | psoriasis) |
| | | | Inflammatory bowel diseases, such as |
| | | | Crohn’s disease and ulcerative colitis |
| | | | ARDS |
| Interleukins | IL-1α | −18.2% | ARDS |
| | IL-1β | −12.0% | Hyperphagia disorders such as Prader- |
| | | | Willi Syndrome and other monogenic and |
| | | | syndromic obesity disorders including |
| | | | leptin pathway deficiencies |
| | | | ARDS |
| | | | Inflammatory bowel diseases, such as |
| | | | Crohn’s disease and ulcerative colitis |
| | IL-3 | −74.5% | ARDS |
| | IL-5 | −29.1% | Type 2 diabetes group (metabolic |
| | | | syndrome, obesity, hyperglycemia) |
| | | | Inflammatory bowel diseases, such as |
| | | | Crohn’s disease and ulcerative colitis |
| | IL-6 | −34.2% | ARDS |
| | | | Chronic autoimmune inflammatory |
| | | | disorders (rheumatoid arthritis, lupus, and |
| | | | psoriasis) |
| | | | Inflammatory bowel diseases, such as |
| | | | Crohn’s disease and ulcerative colitis |
| | | | Hyperphagia disorders such as Prader- |
| | | | Willi Syndrome and other monogenic and |
| | | | syndromic obesity disorders including |
| | | | leptin pathway deficiencies |
| | | | Metabolome-mediated diseases |
| | | | (atherosclerosis, hypertensin, and |
| | | | congestive heart failure) |
| | | | Type 2 diabetes group (metabolic |
| | | | syndrome, obesity, hyperglycemia) |
| | IL-10 | −92.5% | ARDS |
| | IL-12 (p70) | −28.0% | ARDS |
| | | | Metabolome-mediated diseases |
| | | | (atherosclerosis, hypertension, and |
| | | | congestive heart failure) |
| | | | Chronic autoimmune inflammatory |
| | | | disorders (rheumatoid arthritis, lupus, and |
| | | | psoriasis) |
| | IL-17 | −21.2% | ARDS |
| | | | Chronic autoimmune inflammatory |
| | | | disorders (rheumatoid arthritis, lupus, and |
| | | | psoriasis) |
| | | | Inflammatory bowel diseases, such as |
| | | | Crohn’s disease and ulcerative colitis |
| | | | Metabolome-mediated diseases |
| | | | (atherosclerosis, hypertension, and |
| | | | congestive heart failure) |
| | | | Type 2 diabetes group (metabolic |
| | | | syndrome, obesity, hyperglycemia) |
| IFNs | IFN-γ | −37.3% | Metabolome-mediated diseases |
| | | | (atherosclerosis, hypertension, and |
| | | | congestive heart failure) |
| | | | Type 2 diabetes group (metabolic |
| | | | syndrome, obesity, hyperglycemia) |
| | | | Chronic autoimmune inflammatory |
| | | | disorders (rheumatoid arthritis, lupus, and |
| | | | psoriasis) |
| | | | ARDS |
| | | | Inflammatory bowel diseases, such as |
| | | | Crohn’s disease and ulcerative colitis |

### Microbiome

There were changes in the microbiome in a mice model using a high-fat diet after 4 weeks of treatment with and without administering DA orally. The high fat diet itself induced extensive changes in the microbial populations in all groups. Importantly though, there was a pronounced difference between the DA treatment group and the control group at week 4.

Classifications of the different organisms that changed in the control and treatment groups at 4 weeks and observed extensive changes in the primary or dominant phylum groups of bacteria, as well as on a family and genus level were made. For example, Firmicutes were dramatically reduced in the treatment group while Proteobacteria and Verrucomicrobia were dramatically increased. The diversity at 4 weeks dropped over the study course in both control and treatment group due to dietary impact. The treatment group had further significantly reduced overall diversity compared to control at 4 weeks, indicating an increase in specialized populations.

The genetic potential of treatment-induced changes in relation to predicted physiological and metabolic pathways were aligned with observed benefits of treatment with DA with regards to attenuating inflammation and metabolic syndrome. The majority of the pathways being impacted were directly related to a decrease in inflammation and are known to be beneficial to cardiovascular health and other conditions related to the metabolic syndrome in humans. Observations included:
Increased metabolism of unsaturated fatty acids
Increased metabolism of arachidonic acid
Increased metabolism of cofactors and vitamins
Increased lysine degradation
Increased glycolysis and gluconeogenesis
Increased phosphatidylinositol signaling
Decreased arginine and ornithine metabolism
   Below Changes from Phylum à Family à Genus Level

Genetic Potential 1: Increased metabolism of unsaturated fatty acids. There was a significant enrichment in pathways for biosynthesis of unsaturated fatty acids. Accumulating evidence supports a benefit of dietary unsaturated fatty acids over saturated fatty acids to improve cardiovascular health (Front Pharmacol. 2018; 9:1082; Circulation. 2017; 136(3):e1-e23; Ann. Intern. Med. 2014; 160(6):398-406).

Genetic Potential 2: Increased metabolism of arachidonic acid. Arachidonic acid metabolites are important factors in the initiation and resolution of inflammation, and have been linked to the pathophysiology of obesity, diabetes mellitus, nonalcoholic fatty liver disease (NAFLD)/nonalcoholic steatohepatitis (NASH), and cardiovascular diseases (Int. J. Mol. Sci. 2018; 19(11): 3285).

Genetic Potential 3: Increased metabolism of cofactors and vitamins. Increase in production of cofactors and vitamins have interactive effects. Cofactors, including 1-carnitine, nicotinamide riboside (NR), 1-serine, and N-acetyl-1-cysteine (NAC), have been demonstrated in human clinical studies to improve altered biological functions associated with different human diseases (Nutrients. 2019; 11(7):1578). Multiple vitamins and their derivatives have therapeutic potential for prevention and treatment of metabolic syndrome diseases, including diabetes mellitus (Can. J. Physiol. Pharmacol. 2015; 93(5):355-62; Endocr. Metab. Immune Disord. Drug Targets. 2015; 15(1):54-63).

Genetic Potential 4: Increased lysine degradation. Major end products of lysine degradation are bacterial butyrate (Annu. Rev. Biochem. 1981; 50:23-40), which has been shown to prevent atherosclerosis by maintaining gut barrier function (Nat. Microbiol. 2018; 3(12):1332-1333). Another end product, acetate, has also similar effects to reduce inflammation (J. Atheroscler. Thromb. 2017; 24(7):660-672).

Genetic Potential 5: Increased glycolysis and gluconeogenesis. Short chain fatty acid (SCFA) production in bacteria is sequential from glycolysis of glucose to pyruvate, to acetyl coenzyme A (CoA), and eventually to acetic acid, propionic acid, and butyric acid (J. Lipid Res. 2016; 57(6):943-54). This regulation ties in with previously noted pathways including lysine degradation.

Genetic Potential 6: Increased phosphatidylinositol signaling. There was a significant phosphatidylinositol pathway upregulation. It has been documented that phosphatidylinositol pathways (e.g., PI3K/AKT, MAPK and AMPK pathways) are essential for glucose homeostasis. Moreover, deregulation of these pathways often results in obesity and diabetes (Expert Rev. Mol. Med. 2012; 14:e1).

Genetic Potential 7: Decreased arginine and ornithine metabolism. We observed that arginine and ornithine metabolism pathways are significantly reduced. A randomized study proposed that high arginine levels were associated with higher risk of ischemic heart disease (Am. Heart J. 2016; 182:54-61), and accumulation of ornithine is also involved in pathogenesis of several metabolic diseases (Biomed. Pharmacother. 2017; 86:185-194).

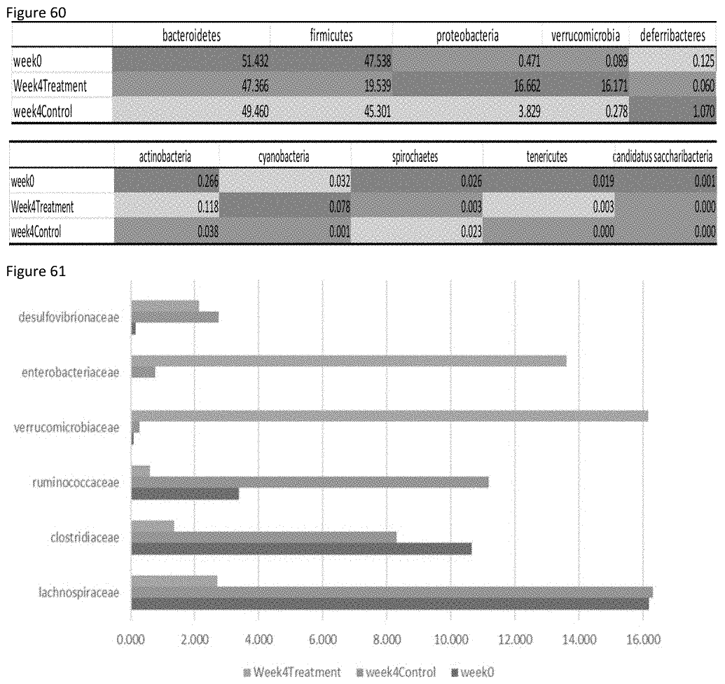

FIG. 60 shows changes a phylum levels wherein week 4 showed>95% confidence changes in the microbiome at the phylum level for the following: Treatment increased proteobacteria*, verrucomicrobia*, cyanobacteria*. Treatment decreased Bacteroidetes, firmicutes*, deferribacteres and spirochetes*.

*significant differences from control or time 0

FIG. 61 shows significant differences for treatment versus control at a family level.
Genus significantly different between treatment at 4 weeks versus baseline and control.
Significantly Increased
Parabacteroides
Escherichia
Erysipelatoclostridium
Peptoclostridium-
Sutterella
Shigella
Brenneria
Significantly Decreased
Lachnoclostridium
Barnesiella
Clostridium
Oscillospira
Dorea
Candidatus soleaferrea
Dehalobacterium
Oscillibacter
Flavonifractor

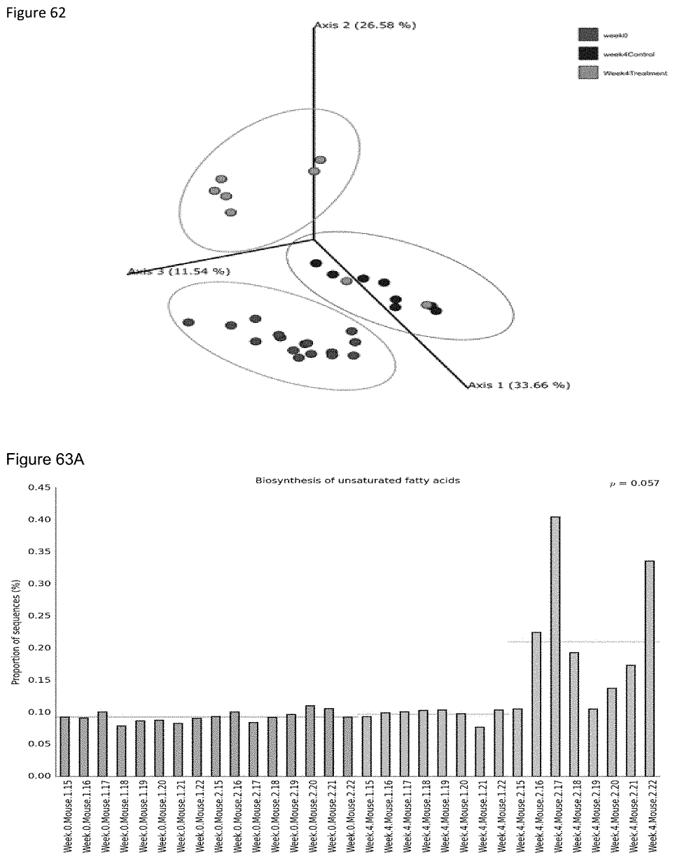

FIG. 62 shows a principal coordinate analysis plot.

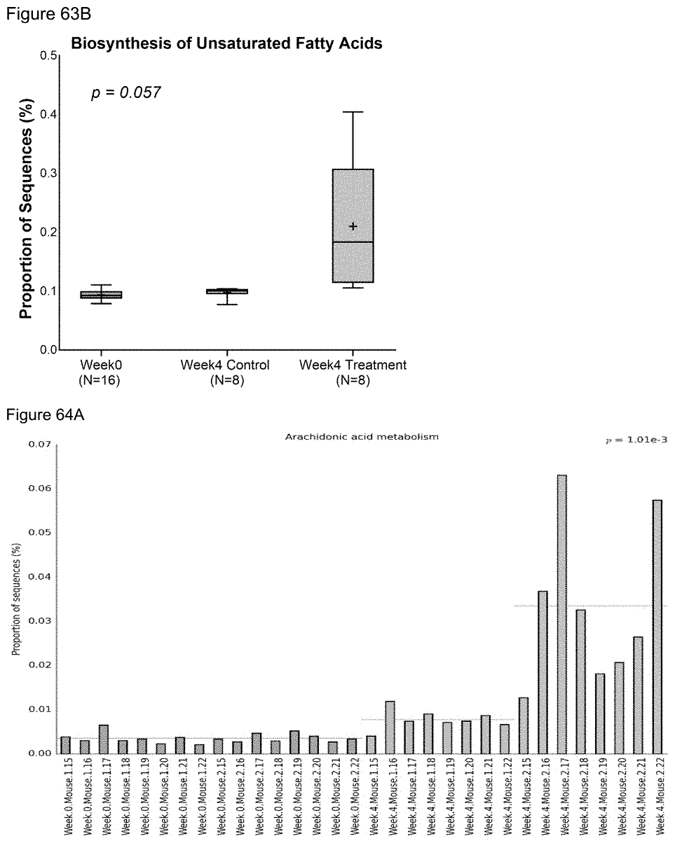

FIG. 63 shows a significant enrichment in the pathways for biosynthesis of unsaturated fatty acids upon 4-week DA treatment (upper panel: individual data; lower panel: group data).

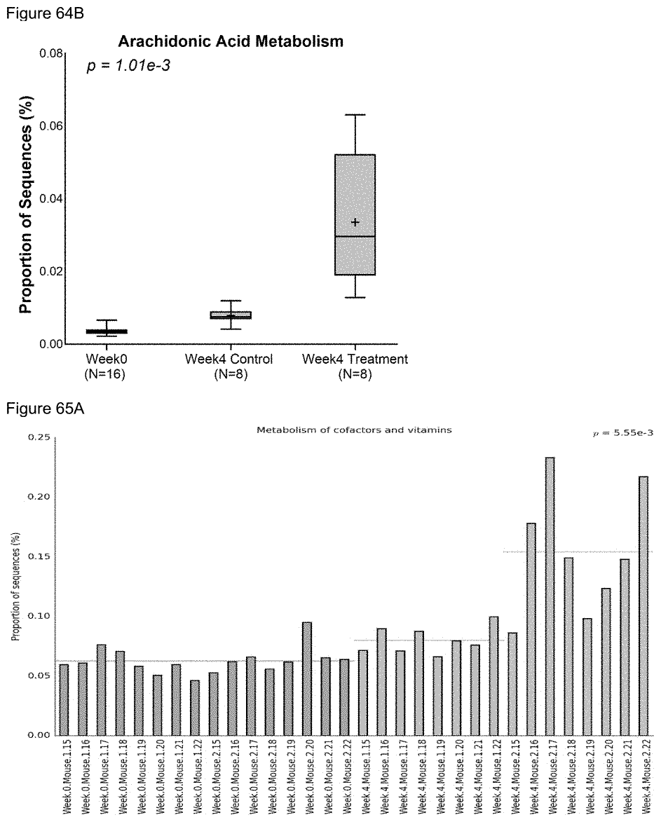

FIG. 64 shows a significant enrichment in the pathways for metabolism of arachidonic acid upon 4-week DA treatment (upper panel: individual data; lower panel: group data).

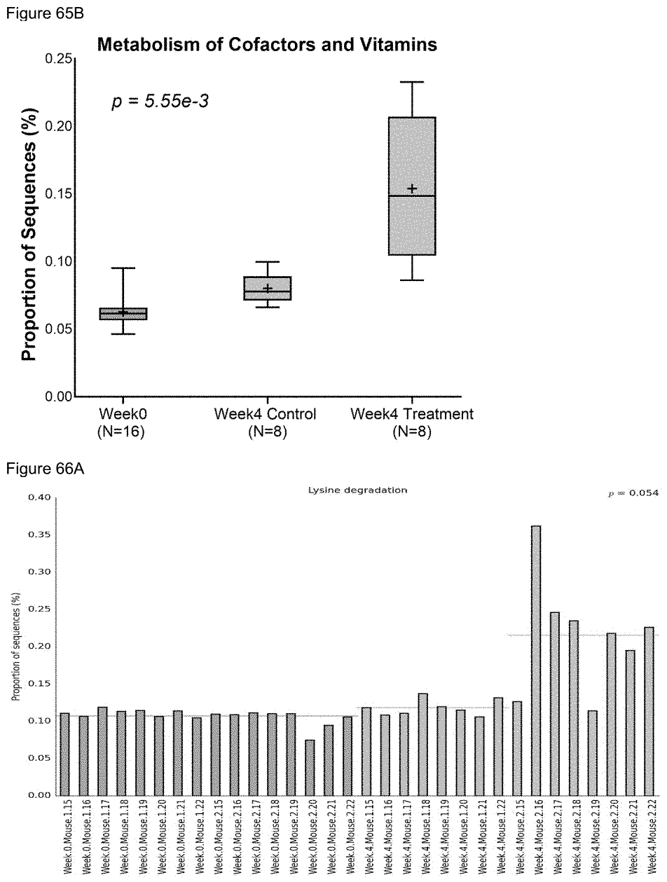

FIG. 65 shows a significant enrichment in the pathways for metabolism of cofactors and vitamins upon 4-week DA treatment (upper panel: individual data; lower panel: group data).

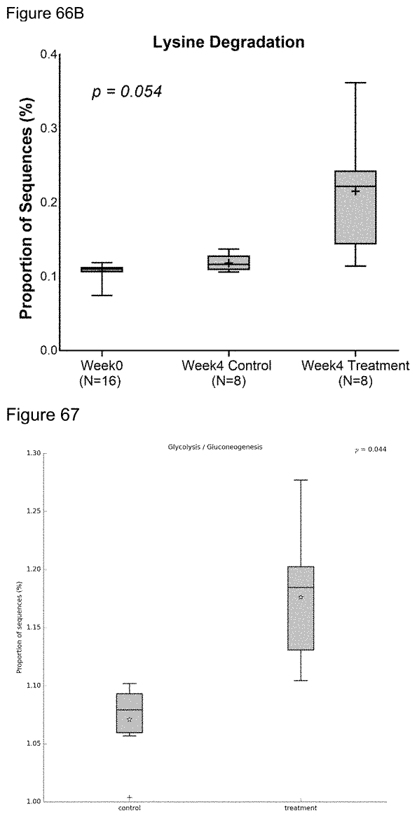

FIG. 66 shows a significant enrichment in pathways for lysine degradation upon 4-week DA treatment (upper panel: individual data; lower panel: group data).

FIG. 67 shows a significant enrichment in pathways for glycolysis and gluconeogenesis upon 4-week DA treatment (group data).

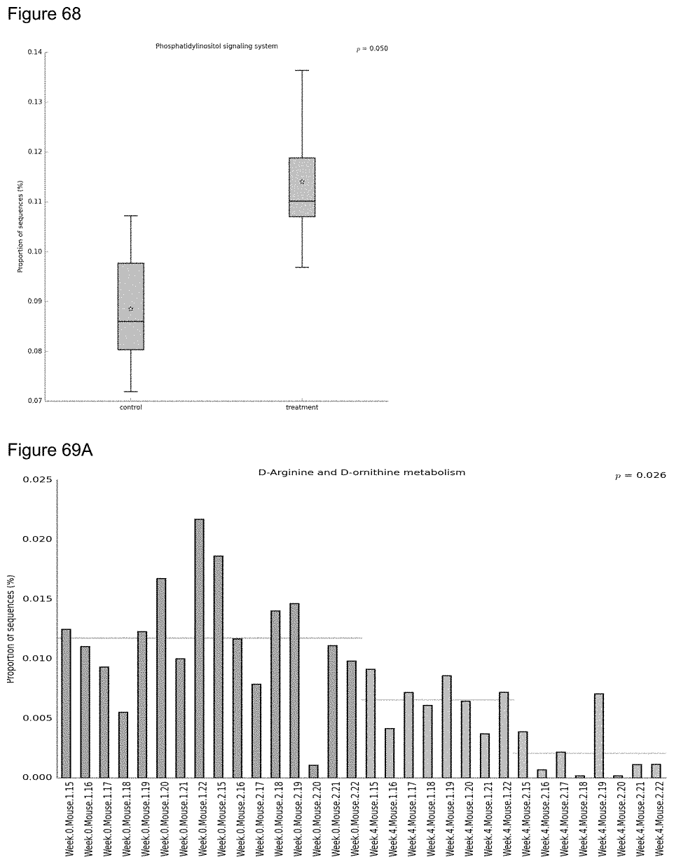

FIG. 68 shows a significant enrichment in phosphatidylinositol signaling upon 4-week DA treatment (group data).

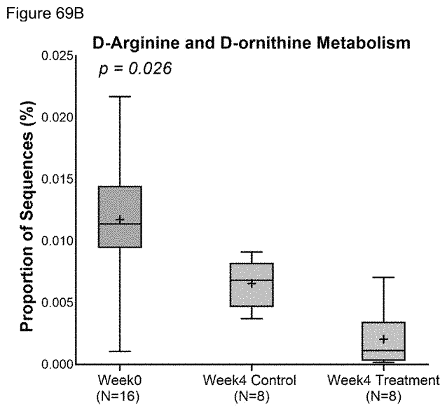

FIG. 69 shows a significantly decreased signaling for arginine and ornithine metabolism upon 4-week DA treatment (upper panel: individual data; lower panel: group data).

### Example 1

This example describes an in vivo study of denatonium acetate on body weight in leptin-deficient (ob/ob) mice. Adult leptin-deficient mice (homozygote, ob/ob mice) fed with high-fat diet. There was a vehicle control group (15 mice) that were treated with distilled water by gavage BID. The DA group (15 mice) were treated with a DA solution at a dose of 23.1 mg/kg BID.

Body weights and body weight changes were determined at days 1, 3, 7, 10, 14, 21, 24 and 28. Food intake was determined on days 3, 7, 10, 14, 17, 21, 14 and 28. On day 28 blood samples were taken for cytokine analysis, HbA1c, HDL, LDL, insulin, and bile acids. Statistics were done by two-way repeated measures ANOVA followed by Tukey's multiple comparison post hoc test.

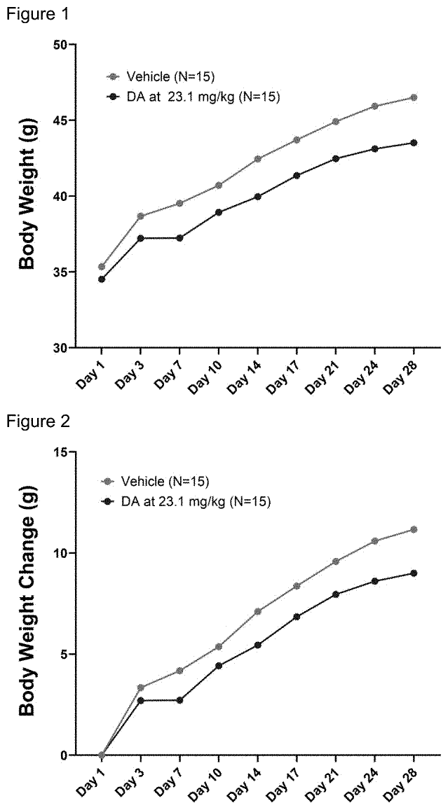

Table 3A and FIG. 1 show body weight measurements from days 1-28.

TABLE 3A

| ANOVA table | SS | DF | METS | F (DFn, DFd) | P value |
|---|---|---|---|---|---|
| Time × | 28.21 | 8 | 3.527 | F (8, 224) = 1.833 | P = 0.0721 |
| Treatment | | | | | |
| Time | 2775 | 8 | 346.9 | F (1.210, 33.89) = | P < 0.0001 |
| | | | | 180.3 | |
| Treatment | 314.9 | 1 | 314.9 | F (1, 28) = 2.053 | P = 0.1630 |
| Subject | 4296 | 28 | 153.4 | F (28, 224) = 79.73 | P < 0.0001 |
| Residual | 431.1 | 224 | 1.924 | | |

Drug treatment showed no significant main effect on body weight in ob/ob mice [F (1, 28) = 2.076, P = 0.163].

Table 3B and FIG. 2 show body weight changes from days 1-28.

TABLE 3B

| ANOVA table | SS | DF | METS | F (DFn, DFd) | P value |
|---|---|---|---|---|---|
| Time × | 28.21 | 8 | 3.527 | F (8, 224) = 1.833 | P = 0.0721 |
| Treatment | | | | | |
| Time | 2775 | 8 | 346.9 | F (1.210, 33.89) = | P < 0.0001 |
| | | | | 180.3 | |
| Treatment | 120.0 | 1 | 120.0 | F (1, 28) = 2.809 | P = 0.1049 |
| Subject | 1196 | 28 | 42.72 | F (28, 224) = 22.20 | P < 0.0001 |
| Residual | 431.1 | 224 | 1.924 | | |

Drug treatment showed no significant main effect on body weight change in ob/ob mice [F (1, 28) = 3.849, P = 0.105].

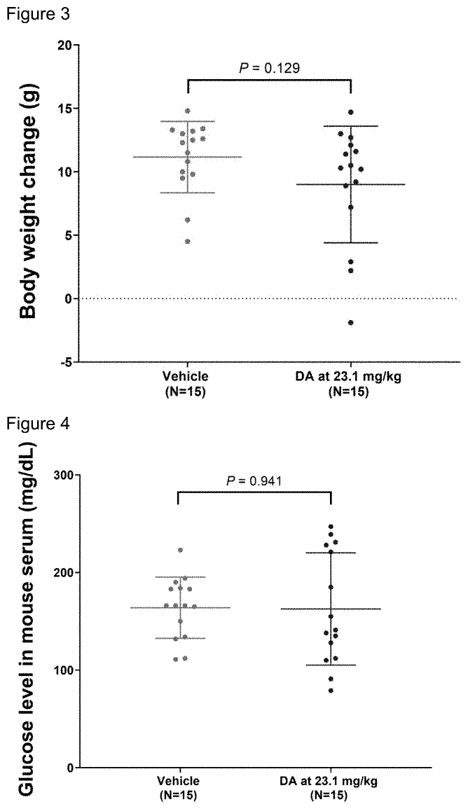

FIG. 3 shows the body weight change at day 28. There was no statistically significant difference in body weight change at day 28 between the two experimental groups.

FIG. 4 shows fasting blood glucose levels at day 28. There was no statistically significant difference in blood fasting glucose level at day 28 between the two experimental groups.

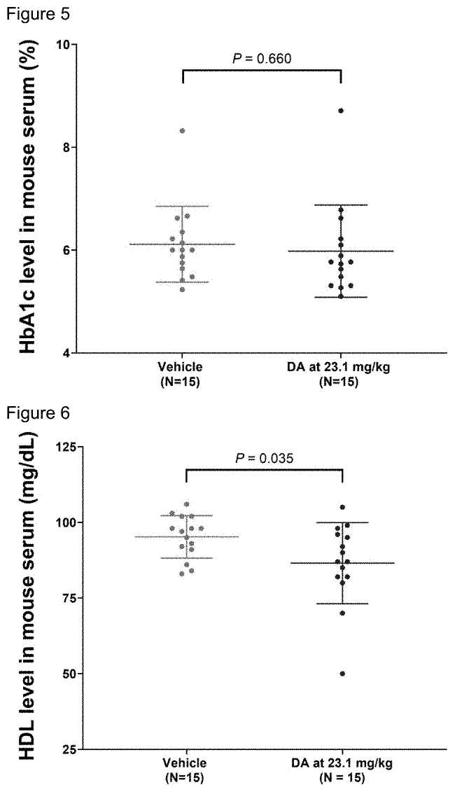

FIG. 5 shows HbA1c levels at day 28. There was no statistically significant difference in blood HbA1c levels at day 28 between the two experimental groups.

FIG. 6 shows blood HDL levels at day 28. Animals treated with DA at 23.1 mg/kg showed a statistically significant decrease in blood HDL level at day 28 compared to vehicle-treated animals.

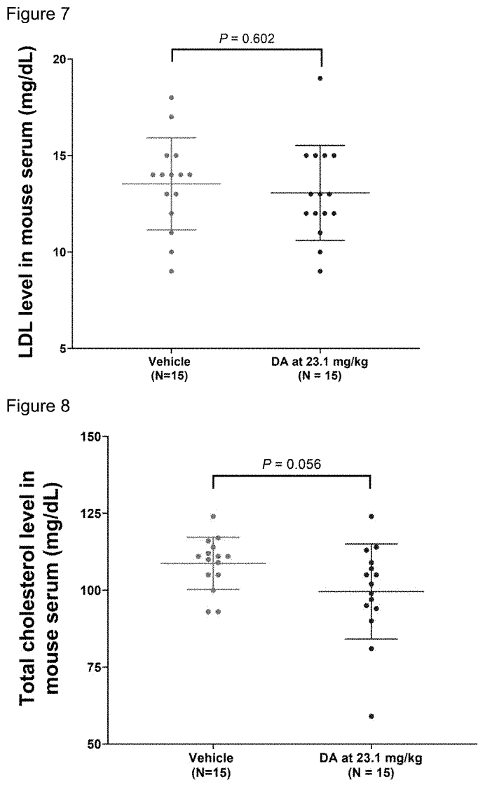

FIG. 7 shows blood LDL cholesterol levels at day 28. There was no statistically significant difference in blood LDL levels at Day 28 between the two experimental groups.

FIG. 8 shows blood total cholesterol level (LDL plus HDL) at day 28. Animals treated with DA at 23.1 mg/kg showed an almost significant decrease in blood total cholesterol levels at day 28 compared to vehicle-treated animals.

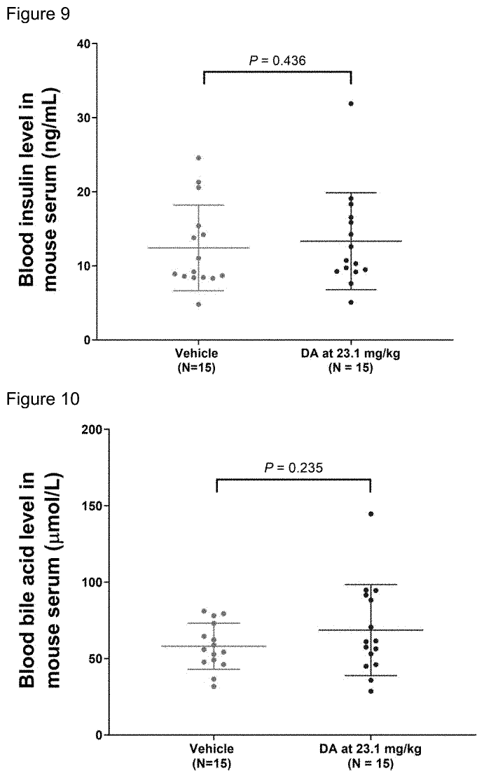

FIG. 9 shows blood insulin levels at day 28. There was no statistically significant difference in blood insulin levels at day 28 between the two experimental groups.

FIG. 10 shows blood bile acid levels at day 28. There was no statistically significant difference in blood bile acid levels at day 28 between the two experimental groups.

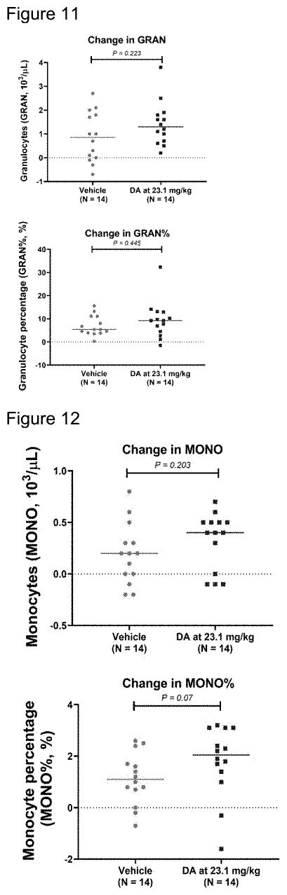

FIG. 11 shows granulocyte number and percentage at pre-dose and at day 28, Although there was no statistically significant difference, DA-treated animals showed a trend of increasing change in granulocyte number as compared to vehicle-treated controls.

FIG. 12 shows monocyte number and percentage at pre-dose and at day 28. Although there was no statistically significant difference, DA-treated animals showed a trend of increasing change in monocyte number and percentage as compared to vehicle-treated controls.

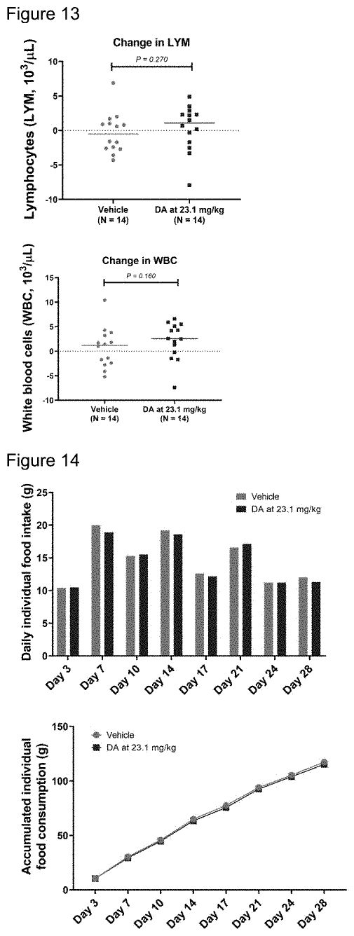

FIG. 13 shows changes in lymphocyte and white blood cell number at pre-dose and at day 28. Although there was no statistically significant difference, DA-treated animals showed a trend of increasing change in lymphocyte and white blood cell numbers and percentage as compared to vehicle-treated controls.

FIG. 14 shows cumulative food consumption over 28 days. There was no statistically significant difference in food consumption over 28 days between the two experimental groups.

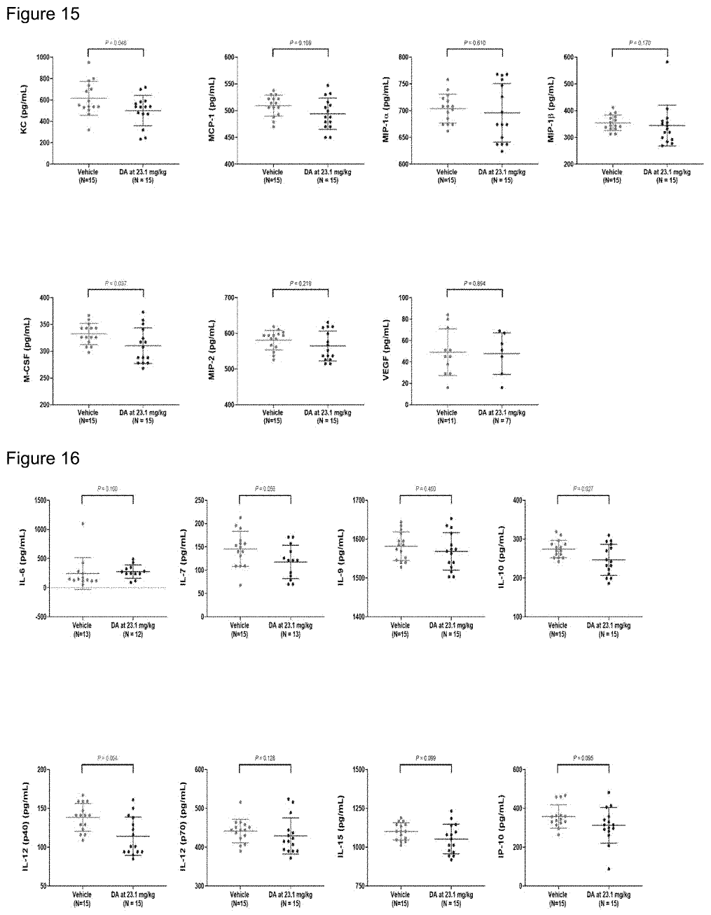

FIG. 15 shows various cytokines analysis in blood at day 28. KC: cytokine-induced neutrophil chemoattractant (CXCL1); MCP-1: monocyte chemoattractant protein-1; MIP-1: macrophage inflammatory protein 1; M-CSF, macrophage colony-stimulating factor; MIP-2: macrophage inflammatory protein 2 (CXCL2); VEGF: vascular endothelial growth factor. KC/CXCL1 and M-CSF showed significant decreases with DA administration.

FIG. 16 shows various cytokines analysis in blood at day 28. IP-10: IFN-γ-Inducible Protein 10 (CXCL10). IL-10 and IL-12 showed significant decreases with DA administration.

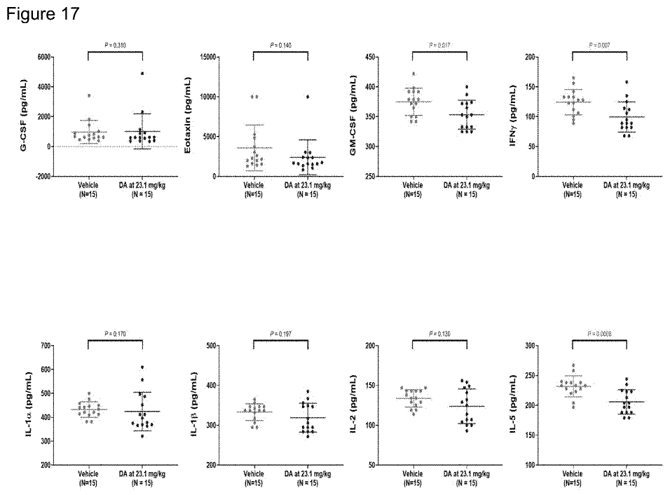

FIG. 17 shows various cytokines analysis in blood at day 28. G-CSF: granulocyte colony-stimulating factor; GM-CSF: granulocyte-macrophage colony-stimulating factor; IFNγ: interferon gamma; IL-1α, IL-1β, IL-2 and IL-5. GM-CSF, IFNγ, and IL-5 showed significant decreases with DA administration.

There is a direct link between chronic inflammation and development of metabolic syndrome and other metabolic disorders (McLaughlin et al. J. Clin. Invest. 2017; 127(1):5-13). Adipose tissue is considered a metabolic risk factor for these medical conditions, and contains a variety of immune cells, including macrophages, eosinophils, innate lymphoid cells (ILCs), T cells, and B cells. This immune cell accumulation induces a chronic low-grade inflammation, influencing metabolism of adipose tissue, promoting systemic inflammation, and impairing insulin action to cause systemic deleterious effects (Wisse, J. Am. Soc. Nephrol. 2004: 15(11):2792-800). Overproduction of proinflammatory factors by this immune cell accumulation has been demonstrated to play a role in this pathogenetic context (Saltiel and Olefsky, J. Clin. Invest. 2017; 127(1):1-4). A wide range of proinflammatory factors, including cytokines and chemokines, show elevated circulating levels in individuals with metabolic syndromes, obesity, diabetes, or other metabolic disorders (Tchernof and Després, Physiol. Rev. 2013; 93(1):359-404). Some proinflammatory factors, like TNF-α or IL-6, have been found to impair insulin action or affect lipid metabolism, thereby contributing to insulin resistance or disordered functions of fat storage (McLaughlin et al. J. Clin. Invest. 2017; 127(1):5-13).

Bitter taste receptors (TAS2Rs) are members of the G protein-coupled receptor (GPCR) family, and are not only on the tongue but throughout the body (Lu et al. J. Gen. Physiol. 2017; 149(2): 181-197). In this study, we did observe that ob/ob mice treated with DA for 28 days showed a noticeable body weight decrease as compared to vehicle-treated controls; while there was no difference in average daily average individual food intake between these two groups of animals. Nevertheless, in DA-treated mice, a panel of cytokines, including GM-CSF, IFNγ, IL-5, IL-10, IL-12, KC, and M-CSF, showed significant decreases with DA administration. Therefore, the body weight decrease in the DA treatment group may be attributed, at least partly, to the fact that DA-induced agonism at TAS2Rs on the immune cells inhibits the production of these cytokines, subsequently improving inflammation state in the adipose tissues and ameliorating dysfunction of lipid metabolism.

### Example 2

This example provides the results of investigating DA to modulate immune response in a murine air pouch model of inflammation. Eight C57BL/6 mice were assigned to groups for gavage treatment (BID) of controls (distilled water), DA at a dose of 23.1 mg/kg BID (low dose DA), and DA at a dose of 96.4 mg/kg BID (high dose DA). What was measured was infiltrating cell counts with air pouch exudates, IL-6 levels in air pouch exudates by an ELISA assay (R&D Systems Cat. No. M6000B), and multiple cytokine analysis (Mouse 32Plex Kit MilliporeSigma Cat. No. MCYTMAG70PMX32BK). Statistical analysis was done by a one-way ANOVA followed by Tukey's multiple comparison post hoc test for data with normal distribution, Kruskal-Wallis test followed by Dunn's multiple comparison post hoc test for data with skewed distribution, and the ROUT method for identifying outliers.

Duarte et al., Current Protocols in Pharmacology, 5.6.1-5.6.8 Mar. 2012, describes “The subcutaneous air pouch is an in vivo model that can be used to study acute and chronic inflammation, the resolution of the inflammatory response, and the oxidative stress response. Injection of irritants into an air pouch in rats or mice induces an inflammatory response that can be quantified by the volume of exudate produced, the infiltration of cells, and the release of inflammatory mediators. The model presented in this unit has been extensively used to identify potential anti-inflammatory drugs.” It can be used to study localized inflammation without systemic effects. But in this case the drug was administered orally, by gavage BID. In earlier studies with this model, Romano et al. (1997) showed that dexamethasone (powerful anti-inflammatory steroid with severe side effects) by gavage decreased TNF levels.

Test administration was 5 ml/kg body weight BID dosing with 8 hour intervals. The air pouch was created in each test BL6 mouse by sc injection of 1.5 ml/mouse of sterile air on day 0 and 1.5 ml/mouse of sterile air on day 3. Compounds (or control distilled water) were administered BID on day −2. LPS (0.75 mg/animal in 1 ml endotoxin free PBS) was administered at hour 0 or one hour after dosing with test compounds. Plasma samples were collected at termination and exudates of the air pouches for all groups. Cell count analysis and IL-6 assays were conducted at the animal facility and plasma and exudate samples were sent out for cytokine analysis. Each group of distilled water control, 23.1 mg/kg DA and 92.4 mg/kg DA had 8 mice each.

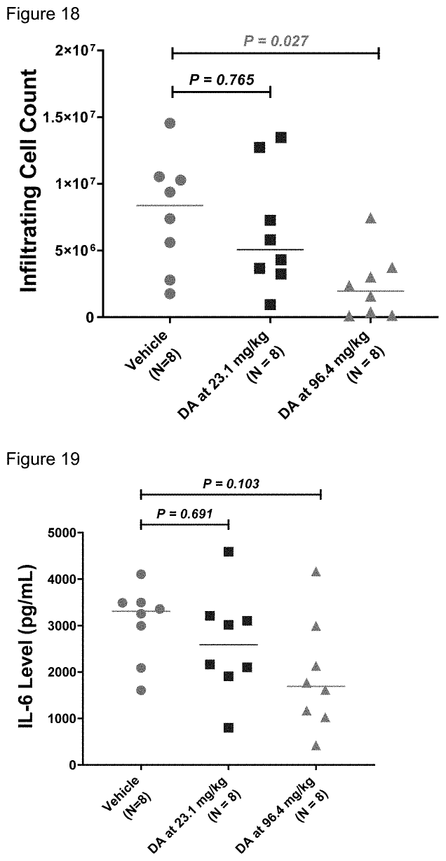

FIG. 18 shows a figure of infiltrating cell counts in air pouch exudates wherein pre-treatment with DA decreased infiltrating cell counts in air pouch exudates following LPS induction in a dose-dependent manner. Animals were pre-treated with DA at 96.4 mg/kg showed significantly lower infiltrating cell count as compared with those pre-treated with vehicle and the lower dose of DA between the results.

FIG. 19 shows a figure of IL-6 levels in air pouch exudates wherein pre-treatment with DA decreased infiltrating cell counts in air pouch exudates following LPS induction in a dose-dependent manner. Animals were pre-treated with DA at 96.4 mg/kg showed significantly lower IL-6 levels as compared with those pre-treated with vehicle and the lower dose of DA between the results.

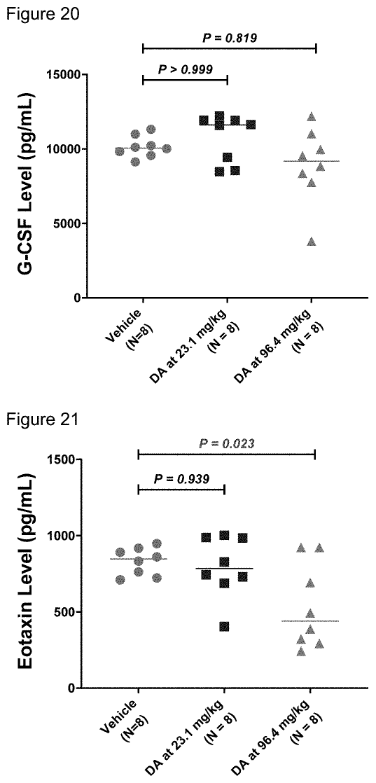

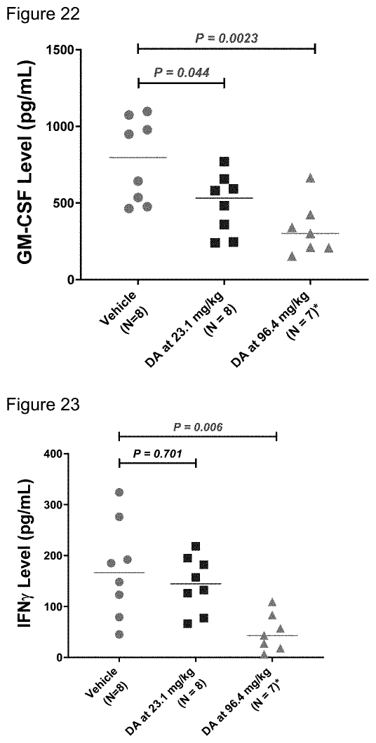

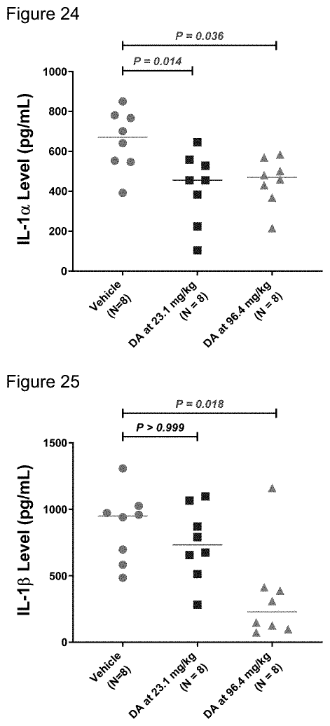

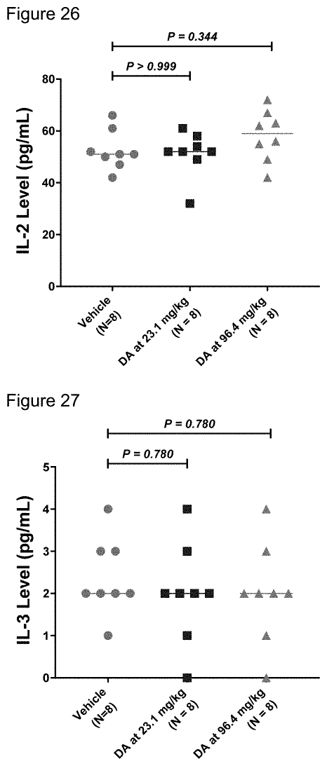

FIGS. 20-27 shows the cytokines levels for G-CSF, Eotaxin, GM-CSF, IFNg, IL-1a, IL-1β. IL-2, and IL-3, respectively. In this group of cytokines, IL-10 showed significant reduction with the higher dose of DA.

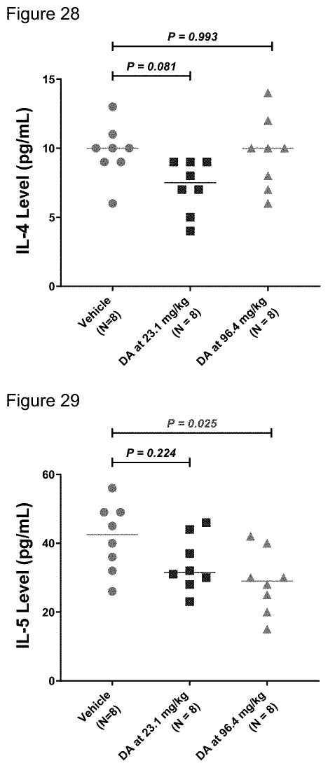

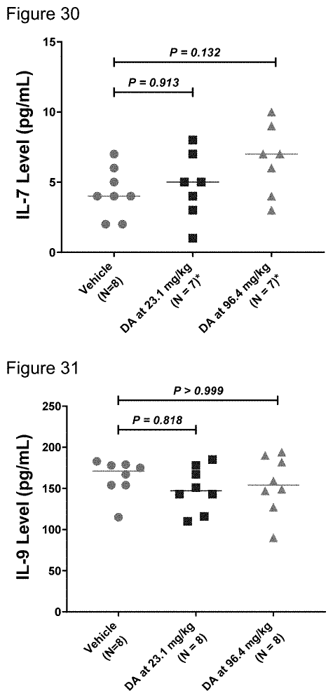

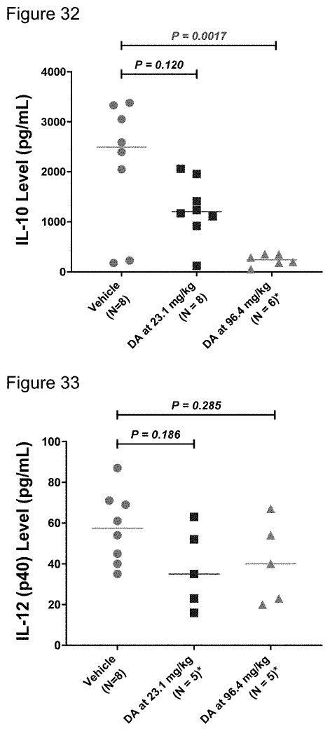

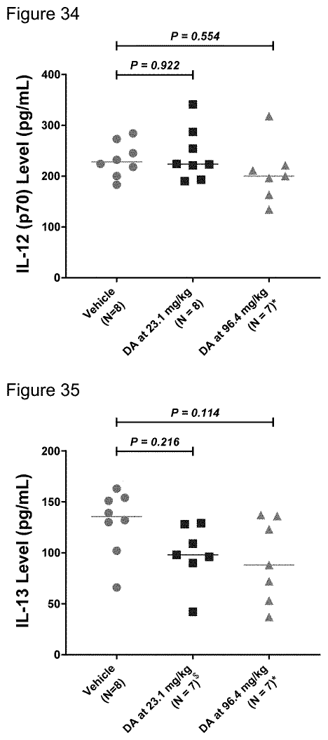

FIGS. 28-35 shows the cytokines levels for IL-4, IL-5, IL-7, IL-9, IL-10, IL-12p40, IL-12p70, and IL-13, respectively. In this group of cytokines, IL-10 showed significant reduction with the higher dose of DA.

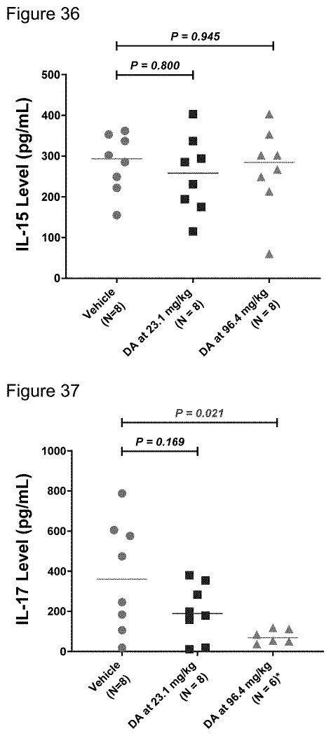

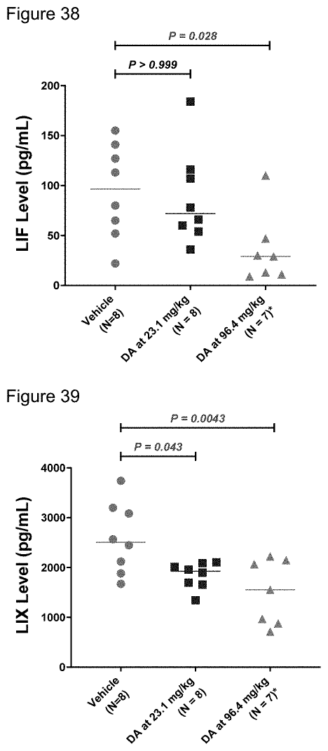

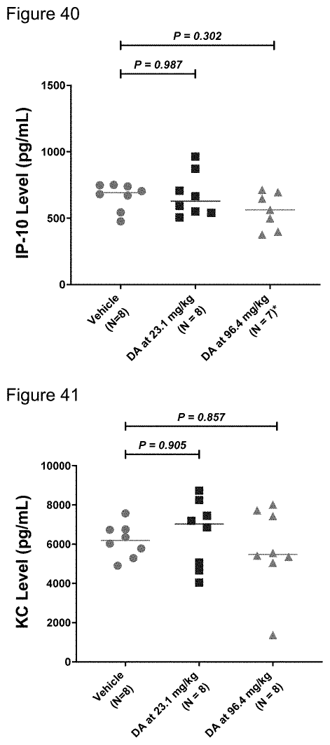

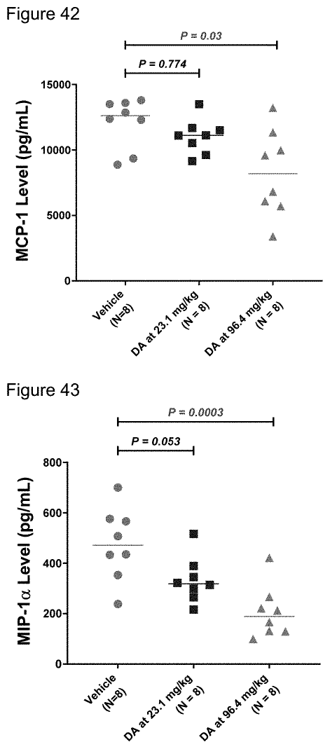

FIGS. 36-43 shows the cytokines levels for IL-15, IL-17, LIF, LIX, IP-10, KC, MCP-1, and MCP-1α, respectively. In this group of cytokines, IL-17 showed significant reduction with the higher dose of DA.

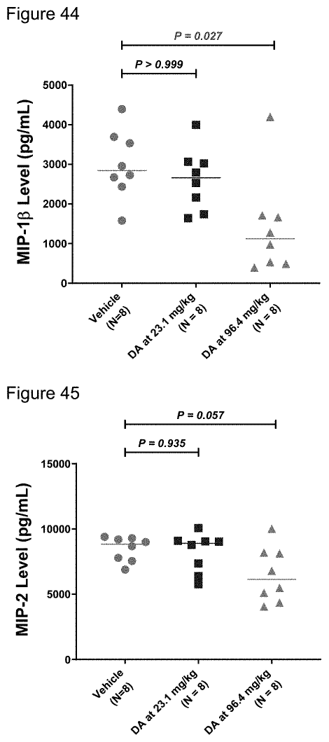

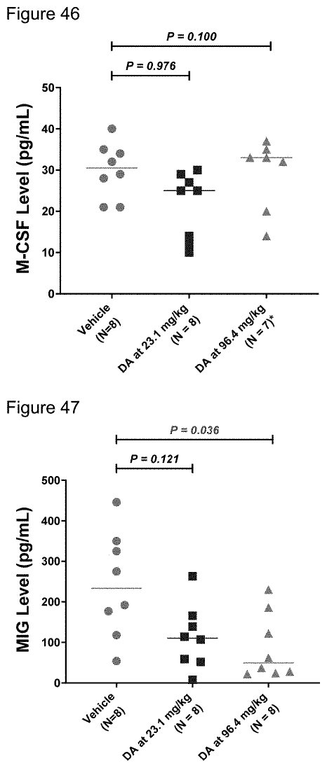

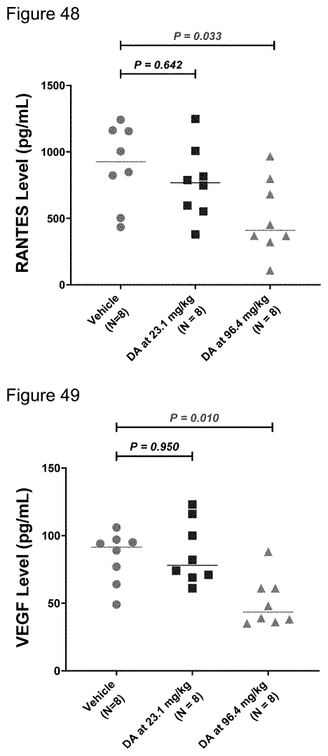

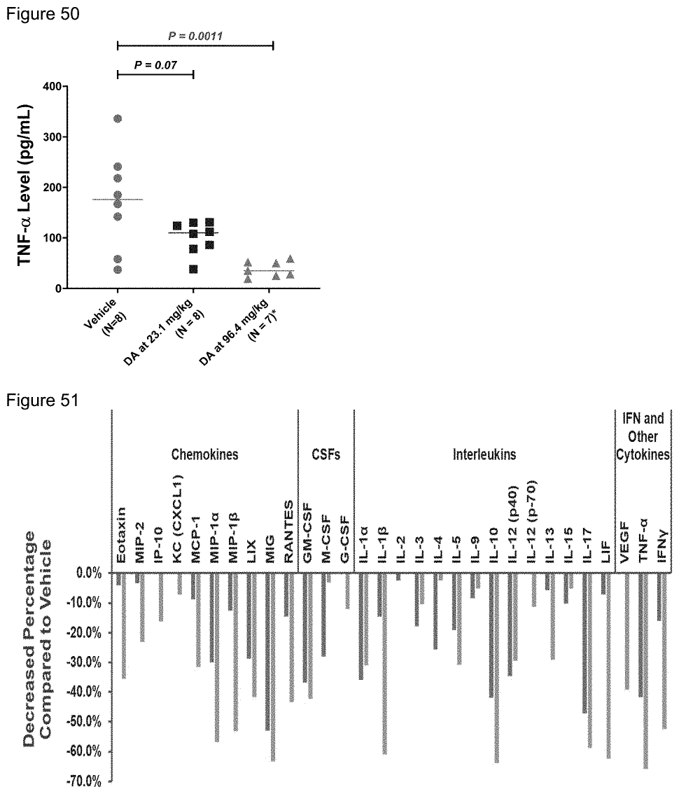

FIGS. 44-50 shows the cytokines levels for MIP-1b, MIP-2, M-CSF, MIG, RANTES, VEGF. and TNF-1a, respectively. In this group of cytokines, TNF-1a showed significant reduction with the higher dose of DA.

In summary, FIG. 51 shows a summary for the higher dose (orange) and the lower dose (blue) showing significance when demarcated with an asterisk. Moreover, the pro-inflammatory biomarkers TNFα, IL-10, IL-10 and IL-17 showed significant dose-response reduction at the higher dose DA administration.

### Example 3

This example provides the results of an in vivo study in a dextran sulfate sodium (DSS)-induced colitis in mice model. Inflammatory bowel diseases (IBD), mainly comprising ulcerative colitis and Crohn's Disease, are complex and multifactorial diseases with unknown etiology. To study human IBD mechanistically, a number of murine models of colitis have been developed. These models are tools to decipher underlying mechanisms of IBD pathogenesis as well as to evaluate potential therapeutics. Among various chemically induced colitis models, the dextran sulfate sodium (DSS) induced colitis model is widely used because of its many similarities with human ulcerative colitis. Moreover, many existing IBD-approved drugs have been studied in this model to allow a comparison of new potential drug compounds as compared with existing drugs with approved IBD indications.

C5BL/6 mice were divided into 5 groups of 3-10 mice, provided with standard mouse chow diet ad libitum, and housed up to 5 per cage. Dexamethasone 21-phosphate disodium salt (DMS; Alfa Aesar Catalog #J64083-1G, Lot R02F035) (was used as a positive control. Hemoccult kits were obtained from Beckman (Hemoccult SENSA kit). Dextran sodium sulfate (DSS) reagent grade (MPI Catalog #160110, Lot #6046H, MW 36,000-50,000, CAS 9011-18-1) was supplemented in the water of certain groups to induced IBD-like symptoms. On day −3 treatment began prior to DSS delivery. On day 1 all mice were pre-weighed and given fresh 4-5% DSS in water every day for 5 days and water is then given for the remainder of the study to elicit disease. An additional control group was given water (no DSS) for the duration of the study (10 days). Body weight was measured daily, fecal blood status (hemoccult) was measured 3× per week, fecal consistency 3× per week and general health determined daily. Mice were sacrificed on day 10 and serum obtained for cytokine analysis and colon length and weight determined. There were two control groups of water only and DSS without drug treatment. There were two treatment groups at 69.3 mg/kg (n=10) bid and 23.1 mg/kg bid (n=10).

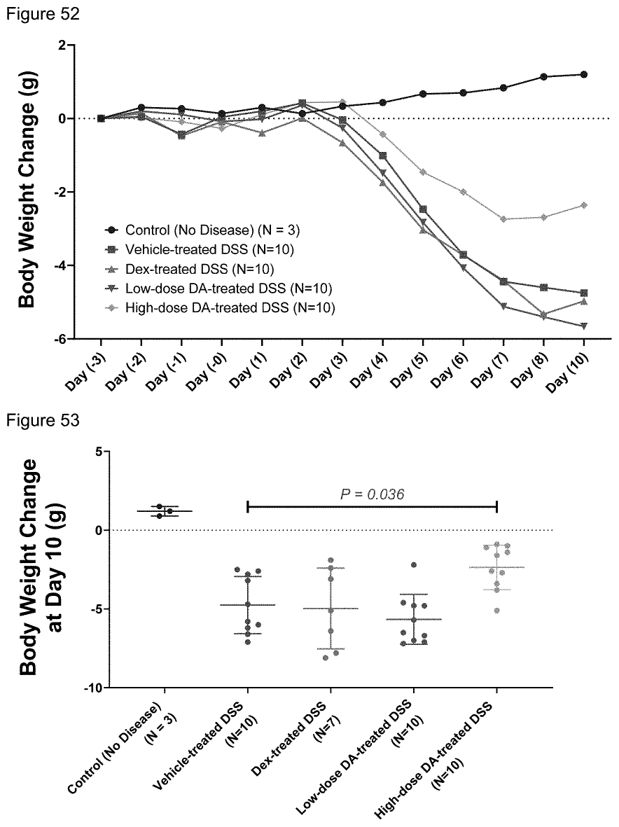

FIG. 52 shows body weight changes during the study period. Treatment with DA showed a significant main effect on body weight (P=0.0052).

FIG. 53 shows body weight at day 10. Animals treated with 69.3 mg/kg DA, BID showed significant effect against DSS-induced body weight loss, as compared to vehicle.

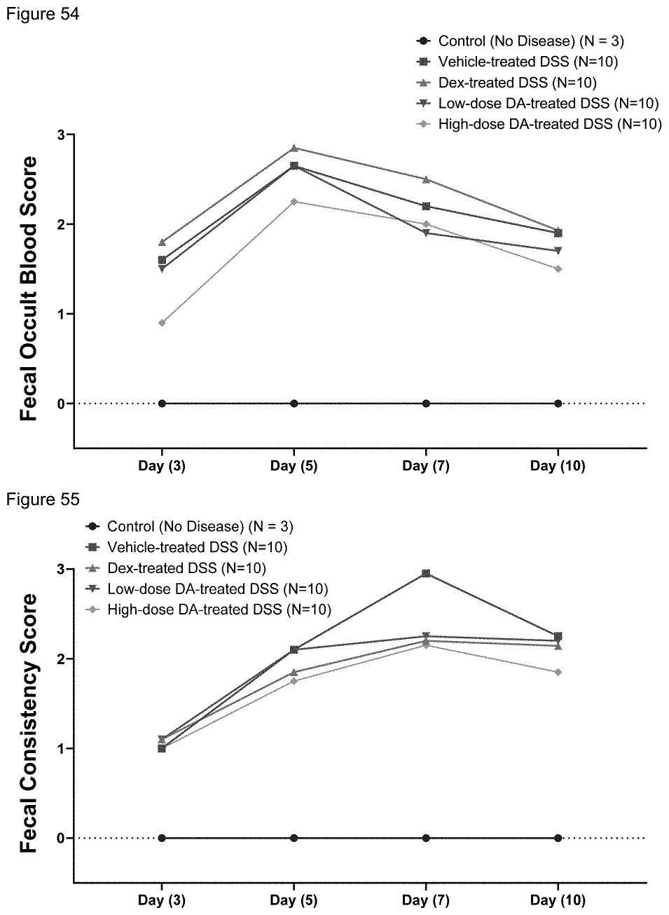

FIG. 54 shows fecal occult blood scores during the study period. Treatment with DA showed a significant main effect on fecal blood status.

FIG. 55 shows fecal consistency score during the study period. Treatment with DA showed significant main effect on fecal consistency.

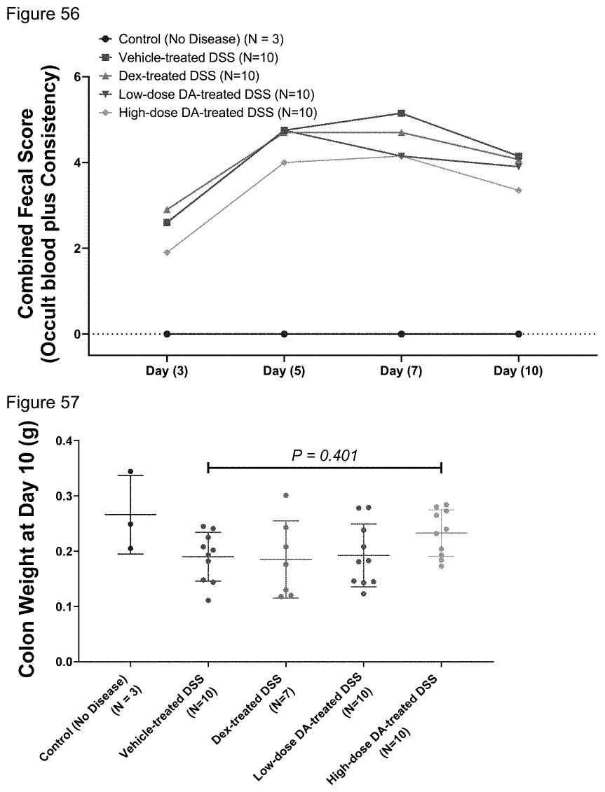

FIG. 56 shows the combined fecal score during the study period. Treatment with DA showed a significant main effect on combined fecal status.

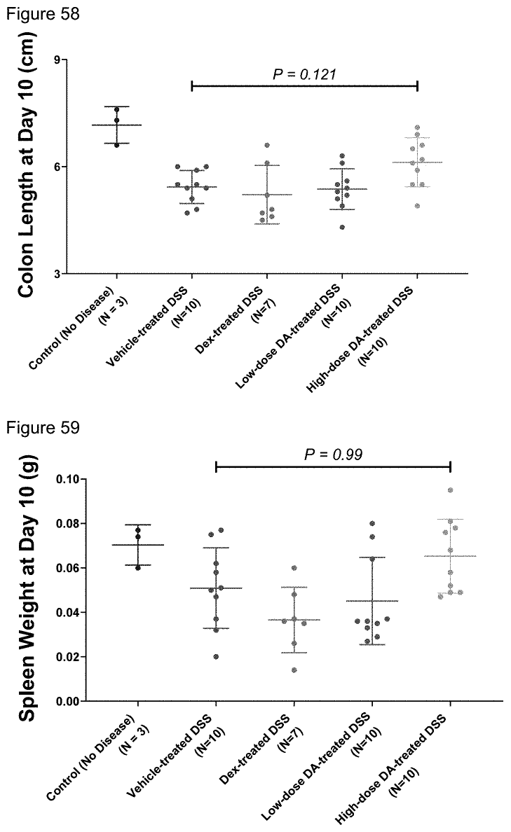

FIGS. 57 and 58 shows colon weight and length at day 10, respectively. Although no significant difference was observed, treatment with high-dose of DA could counteract DSS-induced decrease in colon weight and length in mice.

FIG. 59 shows spleen weight at day 10. Although no significant effect was observed, treatment with high-dose of DA showed a trend to counteract DSS-induced spleen weight loss in mice.

### Example 4

In microbiome studies, low levels of Parabacteroides (protective commensal bacteria) correlate with atherosclerosis, higher Escherichia lead to coronary heart disease (CHD), Ruminococcaceae are often increased in patients with ACVD (atherosclerotic cardiovascular disease), and microbial-produced short chain fatty acids (SCFAs) lead to reduced atherosclerosis, inflammation, and moderate hypertension.

The effect of a small molecule oral TAS2R agonist (DA) was investigated on microbial populations in a nonalcoholic steatohepatitis (NASH) mouse model. Two groups of 4-week-old male C57BL/6 mice (20/group) were fed Amylin Liver NASH (AMLN) diet and received daily doses of ARD-101 (30 mg/mL in water) or vehicle (water) via intragastric gavage. DNA was isolated from fecal samples collected at week 0 and 4, and microbial ecology was evaluated using bTEFAP (bacterial tag-encoded FLX amplicon pyrosequencing). Operational taxonomic units were classified using BLAST against a curated NCBI database. Diversity within specific ecosystems and microbial community structures was analyzed with Qiime 2. Differences were determined by repeated measures ANOVA and post hoc pairwise comparisons using Tukey's test. Taxonomic classification data were evaluated with a dual hierarchal dendrogram.

The AMLN diet led to changes in microbial populations in both groups at week 4. Significant increases/decreases at the phylum, family, and genus levels were observed in the DA group versus vehicle group at week 4. For example, at the phylum level, there were significant increases in Proteobacteria, Verrucomicrobia, and Cyanobacteria and significant decreases in Firmicutes, Deferribacteres, and Spirochetes. There was significantly less diversity within ecosystems and microbial communities at week 4 vs week 0 in both treatment groups and the DA versus vehicle group at week 4 (p<0.05 for all comparisons). Genetic analysis showed that DA led to increased metabolism of unsaturated fatty acids and arachidonic acid, increased production of cofactors and vitamins; increased lysine degradation, glycolysis, gluconeogenesis, and phosphatidylinositol signaling; and decreased arginine and ornithine production. DA treatment-induced significant changes in physiological and metabolic pathways and mitigated the diet-induced decrease of SCFAs in feces. Overall findings are aligned with data showing that DA attenuates inflammation and metabolic syndrome.

### Example 5

This example provides an in vivo study to determine the effect of DA on mouse peritoneum macrophages. Peritoneal exudates were obtained from Balb/c female mice by lavage 4 days after an intraperitoneal injection of 4 ml sterile 4% thioglycollate broth. After washing with RPMI 1640 medium, the cell suspensions were centrifuged at 800 g at 4° C. for 5 min. The red blood cells were eliminated by ACK buffer and the cells were washed and resuspended in RPMI 1640 supplemented with 10% inactivated FBS, 10 mM HEPES, 2 mM glutamine, and 100 U/ml penicillin-100 mg/ml streptomycin. The peritoneal macrophages were plated in 24 well tissue culture plate (2×10⁵ cells/mL/well) at 37° C. in a 5% CO₂ humidified atmosphere. Macrophages were precultured in serum-free RPMI 1640 medium for 24 h to reduce mitogenic effects. Macrophages were pretreated with various concentrations of DA for 1 h prior to LPS treatment and stimulated with LPS (100 ng/mL) for 24 h. Treatment groups were:

TABLE 4

| | No. of | |
|---|---|---|
| Group | Wells | Treatment |
| 1 | 6 | Vehicle |
| 2 | 6 | LPS |
| 3 | 6 | LPS + SB203580 (Positive control) |
| 4 | 6 | LPS + ARD 101 (1 μM) |
| 5 | 6 | LPS + ARD 101 (10 μM) |
| 6 | 6 | LPS + ARD 101 (100 μM)) |

At 12 and 24 h time points of stimulation, ~200 ul of supernatant were removed and stored (−80° C.) for cytokine analysis (13 Plex). Cytokines analyzed were—GM-CSF, IFNγ, IL-1α, IL-1β, IL-2, IL-4, IL-5, IL-6, IL-7, IL-10, IL-12 (p70), IL-13, IL-17A, KC/CXCL1, LIX, MCP-1, MIP-2, TNF-α.

TABLE 5
reports the mean ± SD for each cytokine:

| | Concen- | | |
|---|---|---|---|
| | tration | Significance @12 | Significance @24 |
| cytokine | DA (μM) | hour LPS incubation | hour LPS incubation |
| GM-CSF | 1/10/100 | P = 0.029/no sig/no sig | No sig/no sig/no sig |
| IFNγ | 1/10/100 | P = 0.031/0.037/no sig | No sig/no sig/no sig |
| IL-1α | 1/10/100 | P = 0.021/0.036/no sig | No sig/no sig/no sig |
| IL-1β | 1/10/100 | P = 0.023/0.023/no sig | No sig/no sig/no sig |
| IL-2 | 1/10/100 | P = 0.005/0.004/no sig | No sig/no sig/no sig |
| IL-4 | 1/10/100 | P = 0.009/0.009/no sig | No sig/no sig/no sig |
| IL-6 | 1/10/100 | P = 0.096/0.029/no sig | No sig/no sig/no sig |
| IL-7 | 1/10/100 | P = 0.024/0.010/010 | No sig/no sig/no sig |
| IL-10 | 1/10/100 | P = 0.045/0.026/0.015 | No sig/no sig/no sig |
| IL-12 (p70) | 1/10/100 | P = 0.017/0.007/0.008 | No sig/no sig/no sig |
| IL-13 | 1/10/100 | P = 0.038/0.019/0.021 | No sig/no sig/no sig |
| IL-17A | 1/10/100 | P = 0.044/0.024/0.042 | No sig/no sig/no sig |
| KC/CXCL1 | 1/10/100 | No sig/no sig/P = 0.022 | No sig/no sig/no sig |
| LIX | 1/10/100 | No sig/no sig/no sig | No sig/no sig/no sig |
| MCP-1 | 1/10/100 | No sig/no sig/no sig | No sig/no sig/no sig |
| MIP-2 | 1/10/100 | P = 0.081/0.021/0.033 | No sig/no sig/no sig |
| TNF-α | 1/10/100 | P = 0.059/0.024/0.033 | No sig/no sig/no sig |

In summary, a 24-hour incubatin with LPS dis not elicit the significant differences as a 12 hour LPS incubatin.

### Example 6

This example provides results of a study to evaluate the effect of denatonium acetate on a healthy mouse as measured by cytokine profile and routes of administration of DA. The study groups were: (1) Vehicle group, N=12, treated with distilled water, gavage, BID; (2) DA oral low dose group, N=12, treated with DA at a dose of 23.1 mg/kg (salt weight), gavage, BID; (3) DA oral high dose group, N=12, treated with DA at a dose of 92.4 mg/kg (salt weight), gavage, BID; (4) DA IV low dose group, N=12, treated with DA at a dose of 1 mg/kg (salt weight), iv bolus, QD; (5) DA IV high dose group, N=12, treated with ARD-101 at a dose of 3 mg/kg (salt weight), iv bolus, QD.

Firstly, there were no biomarker (cytokine) effects seen with either iv DA dose. It is safe to conclude that DA needs to be administered orally in order to show effect. Moreover, there were toxic side effects with only iv administration. Group #3 was the lower dose oral DA group and 4 was the higher dose oral DA group, Lower dose DA saw significant decreases in the cytokines (versus controls) for G-CSF (p=0.003), IL-1α (p=0.04), IL-1β (p=0.03), MCP-1 (p=0.005), MIP-2 (p=0.015), and VEGF (p=0.001). Higher dose DA saw significant decreases in the cytokines (versus controls) for GM-CSF (p=0.03), IL-9 (p=0.003), KC (p=0.05), and VEGF (p=0.001). This study confirms biomarker effects in normal mice and confirms that oral dosing, not iv, should be used.

### Example 7

This example provides results of a study to evaluate the effect of denatonium acetate in a mouse acute lung injury plus hyperthermia model. The procedure was three groups of CD-1 mice given (1) saline by gavage for oral administration BID, (2) DA administered oral at a dose of 92.4 mg/kg BID and (3) was DA iv at 3 mg/kg iv bolus QD. Lung lavage fluid was measured and cytokine analysis. Statistics was one-way ANOVA followed by Tukey's multiple comparison post hoc test for data with normal distribution; Kruskal-Wallis test followed by Dunn's multiple comparison post hoc test for data with skewed distribution; and the ROUT method for identifying outliers. Control or drug administered for 3 days, then LPS at 50 μL of 1 mg/ml delivered intratracheally with a Penn Century needle where a core temperature of 39 C at 24 hours post LPS and then sacrifice to measure lung lavage fluid protein concentration and serum cytokine levels.

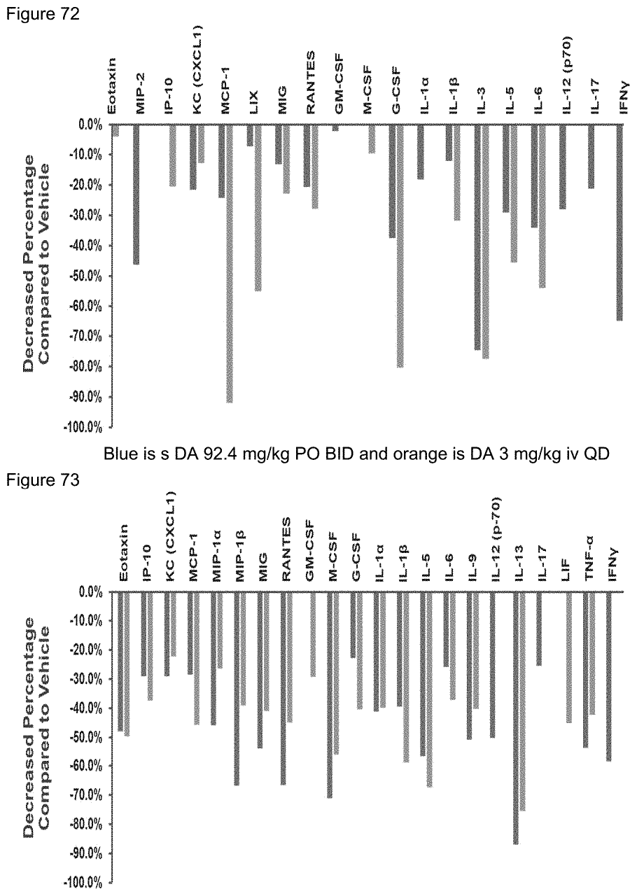

DA showed drastic but not significantly reduced protein concentration in lung lavage fluid for both the oral and iv doses. Cytokine profiles in lung lavage fluids are shown in FIG. 72 where DA=ARD−101.

### Example 8

This example provides results of a second modified acute lung injury plus hyperthermia study to evaluate the effect of denatonium acetate. The same procedure was used as in Example 7. Starting three days before the induction of lung injury, groups of six CD-1 mice each were treated prophylactically with vehicle or 92.4 mg/kg denatonium acetate (DA) (administered by twice-daily (BID) oral gavage (PO)) or with 3 mg/kg DA (administered by once-daily (QD) intraperitoneal (IP) injection). On Day 0, lung injury was induced by intratracheal instillation with 50 μL of 1 mg/mL bacterial lipopolysaccharide (LPS), and hyperthermia was induced by placing the animals in a 39° C. incubator. On Day 1 (i.e., 24 hours after induction), animals were euthanized and bronchoalveolar lavage fluid (BALF) was collected. The BALF specimens were assessed for cytokine concentrations (using a multiplex bead-based assay), and protein levels, and neutrophil counts (by fluorescence-activated cell sorting (FACS)). Additionally, lungs were collected, fixed, stained with Masson's trichrome, and assessed histologically. Three days of repeat PO dosing with 92.4 mg/kg DA (BID) or IP dosing with 3 mg/kg DA (QD) was well-tolerated in female CD-1 mice. Although two mice [one vehicle-dosed, one DA (92.4 mg/kg)-dosed] were found dead on Day 1, the timing of these mortalities (within 24 h after LPS instillation) suggested that the deaths reflected the instillation process, hyperthermia, or associated inflammation (rather than test article). This inference is consistent with the observation that deaths were seen both with vehicle and test article dosing. No other adverse clinical observations were noted during 3 days of test article administration. Oral dosing with 92.4 mg/kg DA yielded significant decreases (compared to vehicle) in the BALF concentrations of 7 of 32 tested cytokines, including IL-2, IL-3, IL-10. IL-13, MCSF, and MIG. IP dosing with 3 mg/kg DA provided significant decreases (compared to vehicle) in the BALF concentrations of 10 of 32 tested cytokines, including G-CSF, eotaxin, IL2, IL-3, IL-4, IL-13, IP-10, MCP-1, M-CSF, and MIG (see FIG. 73). Oral and IP dosing with the indicated levels of DA was associated with nominal (but nonsignificant) changes in BALF protein concentrations; nominal decreases in BALF neutrophil counts (by FACS assay); and nominal decreases in the severity of lung pathology (by histological scoring). Thus, BID PO treatment with 92.4 mg/kg DA or QD IP injection with 3 mg/kg DA provided significant attenuation of the accumulation of multiple cytokines in the lungs of this mouse model of acute lung injury, along with nominal activity in counteracting neutrophil infiltration and lung damage in these animals.

### Example 9

This example provides results of a study of DA plus another compound (CQL) on body weight in diet-induced (DIO) mice. Adult C57BL/6NTac mice were fed with a high fat diet (60%). Vehicle group (N=15) were treated with distilled water by gavage BID, CQL (N=15) were treated at 50 mg/kg by gavage BID, and DA (N=15) at a dose of 92.4 mg/kg by gavage BID. The study period was for 56 days+2-3 days testing period afterward. Body weight change measure 3× per week, food and water consumption on days 0, 12, 28, 42 and 56. Metabolic biomarkers were measured on days 28 and 56. Cytokine analysis on Days 28 and 56. Serum levels of GLP-1, GLP-2, and CCK at 1 h after dosing on Days 1 and 56, and at 2 h after dosing on Day 7 (dosing (>6 h fasting prior to dosing until after blood collection); and serum level of PPY on Day 56.

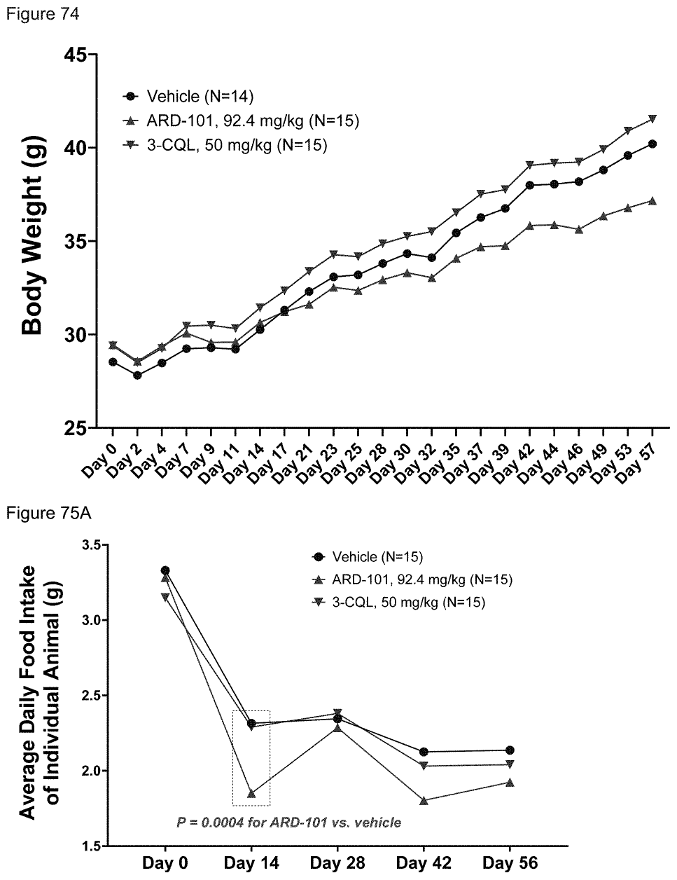

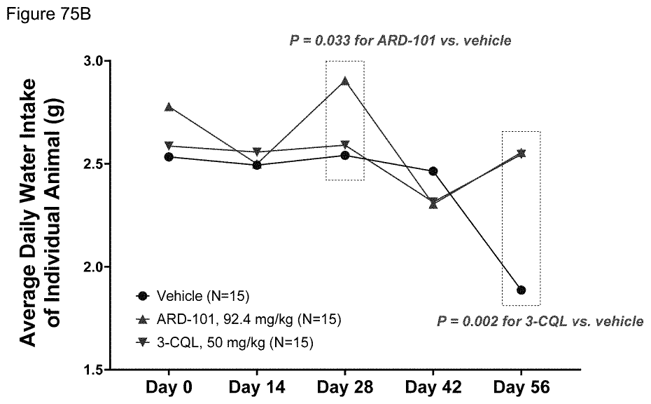

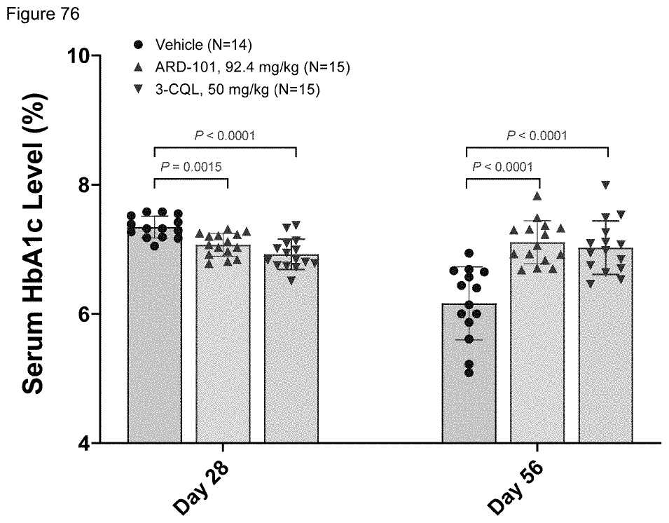

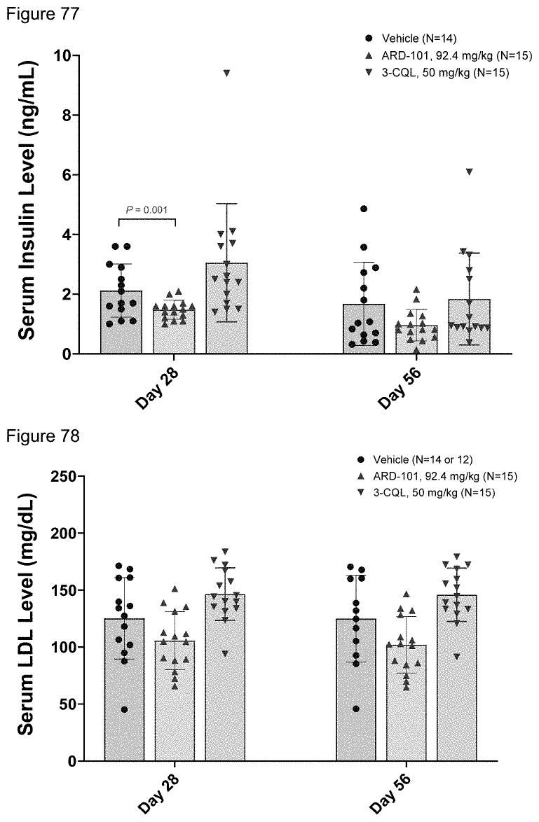

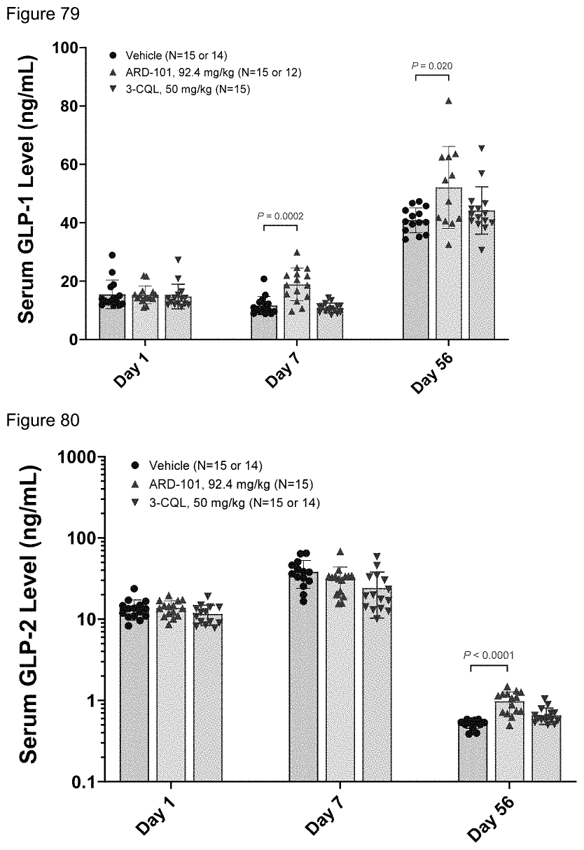

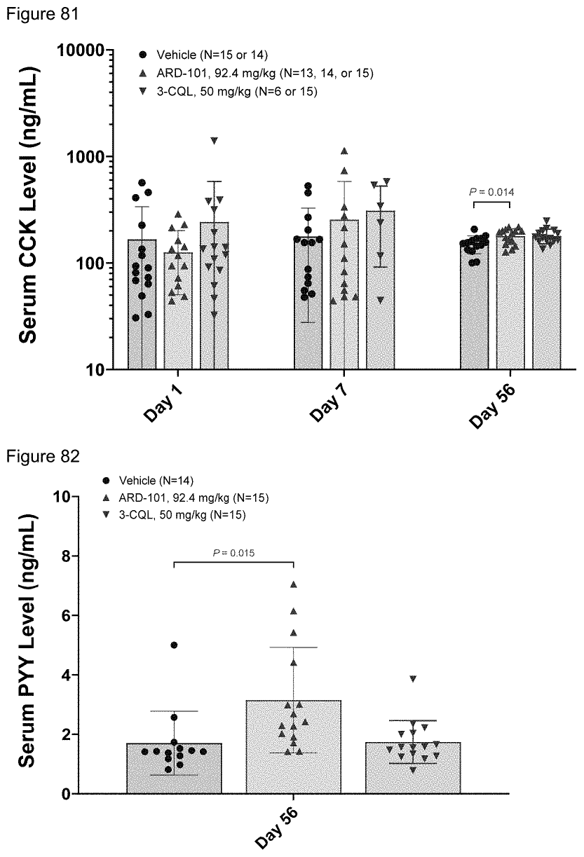

FIG. 74 shows DA treatment significantly reduced body weight gain at day 57 in DIO mice as compared to vehicle and CQL. FIG. 75A shows that at Day 14, treatment with DA significantly reduced daily food intake in DIO mice as compared to vehicle and FIG. 75B shows that treatment with DA significantly increased daily water intake at Day 28, while treatment with CQL significantly decreased daily water intake, as compared to vehicle. Treatment with DA did not show a significant effect on serum glucose levels in DIO mice. FIG. 76 shows that treatments with DA and CQL significantly reduced serum HbA1c level at Day 28, but considerably increased the HbA1c level at Day 56 in DIO mice. FIG. 77 shows that treatments with DA significantly reduced serum insulin level at Day 28 as compared to vehicle control in DIO mice. In FIG. 78 although no significant difference was observed, treatment with DA resulted in noticeable decrease in serum LDL levels at days 28 and 56 as compared to vehicle controls. FIG. 79 shows that treatments with DA significantly increased serum GLP-1 levels in DIO mice at Days 7 and 56 as compared to vehicle control. FIG. 80 shows that treatments with DA significantly increased serum GLP-2 levels in DIO mice at Day 56 as compared to vehicle control. FIG. 81 shows that treatments with DA significantly increased serum CCK levels in DIO mice at Day 56 as compared to vehicle control. FIG. 82 shows that treatments with DA significantly increased serum PYY levels in DIO mice at Day 56 as compared to vehicle control.

At days 28 and 56 (28/56), serum cytokines were measured and showed significant increases for G-CSR (p=0.063/0.039), Eotaxin (p=0.031/no sig), IL-6 (p=0.041/no sig), IP-10 (p=0.013/no sig), and MIG (p=no sig/0.028). Many of the mice did not permit enough blood to be obtained to generate statistical significance.

### Example 10

Leptin-deficient ob/ob mice exhibit hyperphagia and obesity, as well as hyperglycemia and hypertriglyceridemia, which are also found in patients with hyperphagia disorders such as Prader-Willi Syndrome and other monogenic and syndromic obesity disorders (Diabetes. 2006 December; 55(12):3335-43; Clin Genet. 2005 March; 67(3):230-9; Biochim Biophys Acta. 2012 May; 1821(5):819-25). Therefore, ob/ob mice are a predictive in vivo model for these indications. This example provides results of a study of DA plus another compound (CQL) on body weight in leptin-deficient (ob/ob) mice. Vehicle group (N=14) were treated with distilled water by gavage BID, and DA (N=14) at a dose of 50 mg/kg by gavage BID. The study period was for 56 days+2-3 days testing period afterward. Body weight change measured 3× per week, food intake was measure twice per week, metabolic biomarkers (blood glucose, blood insulin, blood HbA1c, HDL, LDL, triglyceride and bile acid) were measured at beginning and end of study. Cytokine analysis was measured at end on Day 56.

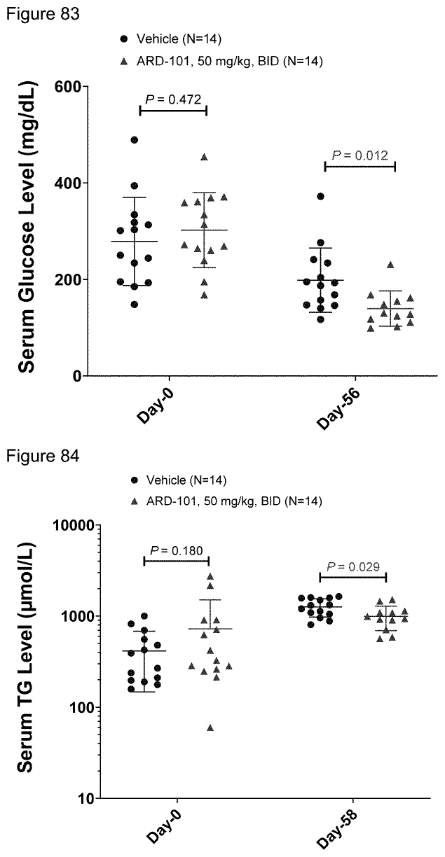

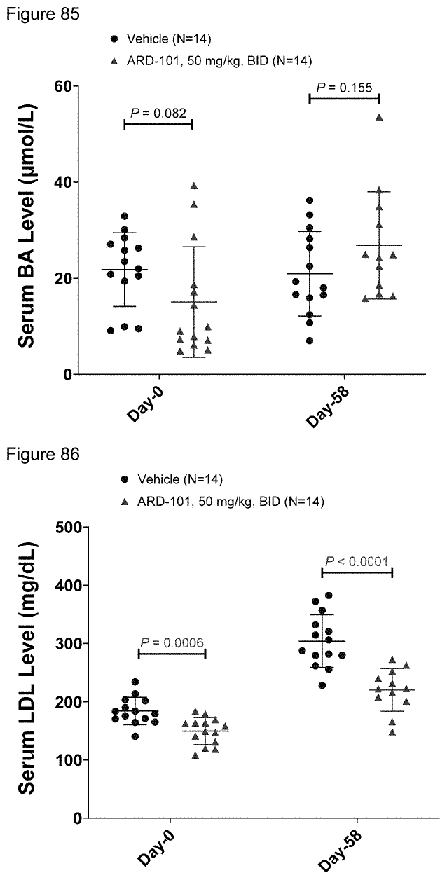

Treatment with DA showed no significant effect on body weight in ob/ob mice. Treatment with DA showed no significant effect on daily food consumption in ob/ob mice. FIG. 83 shows treatment with DA significantly decreased serum glucose levels in ob/ob mice. Treatment with DA showed no significant effect on serum HBA1c levels or insulin levels in ob/ob mice. FIG. 84 shows that treatments with DA significantly lowered serum triglyceride levels as compared to vehicle control in ob/ob mice. FIG. 85 shows that treatments with DA significantly increased serum bile acids levels as compared to vehicle control in ob/ob mice. FIG. 86 shows that treatments with DA significantly lowered serum LDL levels as compared to vehicle control in ob/ob mice. However, there were no significant effects on serum HDL levels.

The DA group saw significant decreases in the cytokines (versus controls) at day 56 for Eotaxin (p=0.047), and MIG (p=0.026). In addition, although no significant difference was observed, the DA group showed decreased levels for the following cytokines at day 56 as compared to the vehicle group: RANTES (decreased by 1.7%), IL-1β (decreased by 19.1%), IL-6 (decreased by 61.4%), and MCP-1 (decreased by 20.9%).

1. A method for treatment, prevention and slowing down exacerbation of type 2 diabetes group of indications selected from the group consisting of metabolic syndrome (METS), obesity, and hyperglycemia, comprising administering orally a pharmaceutic composition comprising a denatonium salt, wherein the denatonium salt is selected from the group consisting of denatonium acetate (DA) denatonium citrate, denatonium maleate, denatonium saccharide, and denatonium tartrate.

2. The method of claim 1, wherein the pharmaceutical composition further comprises from about 0.5 g to about 5 g acetic acid.

3. The method of claim 1, wherein the daily dosage of the denatonium salt for an adult is from about 20 mg to about 5000 mg.

4. The method of claim 3, wherein the daily dosage of DA for an adult is from about 50 mg to about 1000 mg.

5. The method of claim 4, wherein the daily dosage of DA for an adult is from about 60 mg to about 500 mg, or to achieve a concentration in the GI tract of from about 10 parts per billion to about 50 ppm.

6. The method of claim 1, wherein the daily dose of the denatonium salt is administered once per day, twice per day or three times per day.

7. A method for treatment, prevention and slowing down exacerbation of acute pulmonary inflammatory disorders including ARDS, comprising administering orally a pharmaceutic composition comprising a denatonium salt, wherein the denatonium salt is selected from the group consisting of denatonium acetate (DA) denatonium citrate, denatonium maleate, denatonium saccharide, and denatonium tartrate.

8. The method of claim 7, wherein the pharmaceutical composition further comprises from about 0.5 g to about 5 g acetic acid.

9. The method of claim 7, wherein the daily dosage of the denatonium salt for an adult is from about 20 mg to about 5000 mg.

10. The method of claim 9, wherein the daily dosage of DA for an adult is from about 50 mg to about 1000 mg.

11. The method of claim 10, wherein the daily dosage of DA for an adult is from about 60 mg to about 500 mg, or to achieve a concentration in the GI tract of from about 10 parts per billion to about 50 ppm.

12. The method of claim 7, wherein the daily dose of the denatonium salt is administered once per day, twice per day or three times per day.

13. A method for treatment, prevention and slowing down exacerbation of chronic autoimmune inflammatory disorders group of indications selected from the group consisting of rheumatoid arthritis (RA), lupus, and psoriasis, comprising administering orally a pharmaceutic composition comprising a denatonium salt, wherein the denatonium salt is selected from the group consisting of denatonium acetate (DA) denatonium citrate, denatonium maleate, denatonium saccharide, and denatonium tartrate.

14-18. (canceled)

19. A method for treatment, prevention and slowing down exacerbation of chronic inflammatory bowel diseases (IBD) group of indications selected from the group consisting of Crohn's Disease, and ulcerative colitis, comprising administering orally a pharmaceutic composition comprising a denatonium salt, wherein the denatonium salt is selected from the group consisting of denatonium acetate (DA) denatonium citrate, denatonium maleate, denatonium saccharide, and denatonium tartrate.

20-24. (canceled)

25. A method for treatment, prevention and slowing down exacerbation of metabolome mediated group of indications selected from the group consisting of atherosclerosis, hypertension, and congestive heart failure (CHF), comprising administering orally a pharmaceutic composition comprising a denatonium salt, wherein the denatonium salt is selected from the group consisting of denatonium acetate (DA) denatonium citrate, denatonium maleate, denatonium saccharide, and denatonium tartrate.

26-30. (canceled)

31. A method for treatment, or slowing down exacerbation of, hyperphagia group of indications selected from the group consisting of Prader Willi, and leptin pathway deficiencies, comprising administering orally a pharmaceutic composition comprising a denatonium salt, wherein the denatonium salt is selected from the group consisting of denatonium acetate (DA) denatonium citrate, denatonium maleate, denatonium saccharide, and denatonium tartrate.

32. The method of claim 31, wherein the pharmaceutical composition further comprises from about 0.5 g to about 5 g acetic acid.

33. The method of claim 31, wherein the daily dosage of the denatonium salt for an adult is from about 20 mg to about 5000 mg.

34. The method of claim 33, wherein the daily dosage of DA for an adult is from about 50 mg to about 1000 mg.

35. The method of claim 34, wherein the daily dosage of DA for an adult is from about 60 mg to about 500 mg, or to achieve a concentration in the GI tract of from about 10 parts per billion to about 50 ppm.

36. The method of claim 31, wherein the daily dose of the denatonium salt is administered once per day, twice per day or three times per day.
